(19) United States
(12) Patent Application Publication
(10) Pub. No.: US 2011/0065725 A1
(43) Pub. Date: Mar. 17, 2011

(54) TRIAZOLE AMIDE DERIVATIVES FOR USE IN THERAPY

(75) Inventors: Vincenzo Garzya, Essex (GB); Stephen Paul Watson, Essex (GB)

(73) Assignee: GLAXO GROUP LIMITED, Greenford, Middlesex (GB)

(21) Appl. No.: 12/922,101

(22) PCT Filed: Mar. 16, 2009
(86) PCT No.: PCT/EP09/53070

§ 371 Date: Sep. 10, 2010

(30) Foreign Application Priority Data
Mar. 18, 2008 (GB) 0805046.0
Apr. 18, 2008 (GB) 0807162.3
Nov. 20, 2008 (GB) 0821274.8

(51) Int. Cl. A61K 31/4192; C07D 401/12; A61K 31/4439; C07D 403/12; A61K 31/497; C07D 249/06; A61K 31/506; C07D 401/04; A61P 25/00; A61P 25/30

(52) U.S. Cl. 514/255.05; 546/268.4; 514/340; 544/405; 548/255; 514/359; 544/328; 514/256; 544/331; 514/275

(57) ABSTRACT

The invention relates to triazole amide derivatives of formula (I) for use in therapy, in particular for treating diseases and conditions mediated by antagonism of the mGluR₅ receptor, in particular substance related disorders. The invention also relates to certain novel derivatives. In addition, the invention relates to compositions containing the derivatives and processes for their preparation.

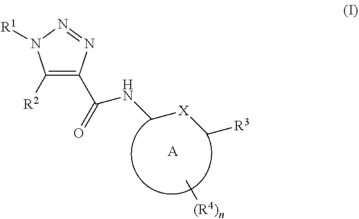

The invention relates to triazole amide derivatives for use in therapy, in particular, for treating diseases and conditions for which antagonism of the mGluR₅ receptor is beneficial in particular substance related disorders. The invention also relates to certain novel derivatives. In addition, the invention relates to compositions containing the derivatives and processes for their preparation.

According to a first aspect, the invention provides a compound of formula (I) or a pharmaceutically acceptable salt thereof, for use in therapy,

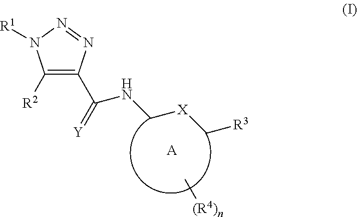

wherein
R¹ is phenyl or 6-membered monocyclic heteroaryl, either of which are optionally substituted by one, two or three groups independently selected from halo, C₁₋₆alkyl, C₁₋₆alkoxy, C₁₋₆haloalkyl, C₁₋₆haloalkoxy, cyano, C₁₋₆alkylthio, C₁₋₆haloalkylthio, C₁₋₆alkylsulfonyl, C₁₋₆haloalkylsulfonyl, C₁₋₆alkylcarbonyl and C₁₋₆haloalkylcarbonyl;
R² is halo, C₁₋₆alkyl, C₁₋₆alkoxy, C₁₋₆haloalkyl, C₁₋₆haloalkoxy or C₁₋₄alkoxyC₁₋₄alkyl;
Y is O or S;
A is either a 6-membered heteroaromatic ring or a benzene ring; wherein X is CH, N, S or O; and wherein when A is a 6-membered heteroaromatic ring, the ring may contain a total of 1 to 4 heteroatoms independently selected from N, O and S;
R³ is halo, C₁₋₆alkyl, C₁₋₆alkoxy, C₁₋₆haloalkyl, C₁₋₆haloalkoxy, C₁₋₄alkoxyC₁₋₄alkyl or cyano;
R⁴ is halo; and
n is 0 or 1.

As used herein unless otherwise indicated, an alkyl substituent is a univalent radical derived by removal of a hydrogen atom from an acyclic alkane. For example, C₁₋₆alkyl refers to such an alkyl substituent containing 1 to 6 carbons. Such alkyl substituents include methyl and ethyl, may be straight chain (i.e. n-propyl, n-butyl, n-pentyl and n-hexyl) or branched chain (for example, isopropyl, isobutyl, secbutyl, tert-butyl, isopentyl and neopentyl). In an embodiment unless otherwise indicated, such an alkyl substituent is methyl, ethyl, n-propyl or isopropyl.

As used herein, a halo substituent refers to fluoro, chloro, bromo and iodo radicals. In an embodiment unless otherwise indicated such a halo substituent is fluoro or chloro.

As used herein unless otherwise indicated, a haloalkyl substituent is an alkyl group substituted by one or more halo substituents, which halo substituents may be the same or different. For example, C₁₋₆haloalkyl refers to such a haloalkyl substituent containing 1 to 6 carbons. Such haloalkyl substituents include monofluoromethyl, difluoromethyl, trifluoromethyl and 1-chloro-2-fluoroethyl. In an embodiment unless otherwise indicated such a haloalkyl substituent is monofluoromethyl, difluoromethyl or trifluoromethyl.

As used herein unless otherwise indicated, an alkoxy substituent is a group of formula “R—O—” where R is alkyl as defined above. For example, C₁₋₆alkoxy refers to such an alkoxy substituent containing 1 to 6 carbons. Such alkoxy substituents include methoxy and ethoxy and may be straight chain (i.e. n-propoxy, n-butoxy, n-pentoxy and n-hexyloxy) or branched chain (for example, isopropoxy, isobutoxy, secbutoxy, tert-butoxy, isopentoxy and neopentoxy). In an embodiment unless otherwise indicated, such an alkoxy substituent is methoxy, ethoxy, n-propoxy or isopropoxy.

As used herein unless otherwise indicated, an alkylthio substituent is a group of formula “R—S—” where R is alkyl as defined above. For example, C₁₋₆alkylthio refers to such an alkylthio substituent containing 1 to 6 carbons. Such alkylthio substituents include methylthio and ethylthio and may be straight chain or branched chain. In an embodiment unless otherwise indicated, such an alkylthio substituent is methylthio, ethylthio, n-propylthio or isopropylthio.

As used herein unless otherwise indicated, an alkylsulfonyl substituent is a group of formula “R—SO₂—” where R is alkyl as defined above. For example, C₁₋₆alkylsulfonyl refers to such an alkylsulfonyl substituent containing 1 to 6 carbons. Such alkylsulfonyl substituents include methylsulfonyl and ethylsulfonyl and may be straight chain or branched chain. In an embodiment unless otherwise indicated, such an alkylsulfonyl substituent is methylsulfonyl, ethylsulfonyl, n-propylsulfonyl or isopropylsulfonyl.

As used herein unless otherwise indicated, a haloalkoxy substituent is a group of formula “R—O—” where R is haloalkyl as defined above. For example, C₁₋₆haloalkoxy refers to such a haloalkoxy substituent containing 1 to 6 carbons. Such haloalkoxy substituents include monofluoromethoxy, difluoromethoxy, trifluoromethoxy and 1-chloro-2-fluoroethoxy and may be straight chain or branched chain. In an embodiment unless otherwise indicated, such a haloalkoxy substituent is monofluoromethoxy, difluoromethoxy or trifluoromethoxy.

As used herein unless otherwise indicated, a haloalkylthio substituent is a group of formula “R—S-” where R is haloalkyl as defined above. For example, C₁₋₆haloalkylthio refers to such a haloalkylthio substituent containing 1 to 6 carbons. Such haloalkylthio substituents include monofluoromethylthio, difluoromethylthio, trifluoromethylthio and 1-chloro-2-fluoroethylthio and may be straight chain or branched chain. In an embodiment unless otherwise indicated, such a haloalkylthio substituent is monofluoromethylthio, difluoromethylthio or trifluoromethylthio.

As used herein unless otherwise indicated, a haloalkylsulfonyl substituent is a group of formula “R—SO₂—” where R is haloalkyl as defined above. For example, C₁₋₆haloalkylsulfonyl refers to such a haloalkylsulfonyl substituent containing 1 to 6 carbons. Such haloalkylsulphonyl substituents include monofluoromethylsulfonyl, difluoromethylsulfonyl, trifluoromethylsulfonyl and 1-chloro-2-fluoroethylsulfonyl and may be straight chain or branched chain. In an embodiment unless otherwise indicated, such a haloalkylsulfonyl substituent is monofluoromethylsulfonyl, difluoromethylsulfonyl or trifluoromethylsulfonyl.

As used herein, unless otherwise indicated, a monocyclic 6-membered heteroaryl substituent refers to a univalent radical derived by removal of a hydrogen atom from a 6-membered monocyclic heteroaromatic ring. The monocyclic 6-membered heteroaryl substituent comprises one or more carbon atoms and 1 to 4 heteroatoms interconnected to form a ring. The heteroatoms are independently selected from nitrogen, oxygen and sulphur. In an embodiment, unless otherwise indicated, the monocyclic 6-membered heteroaryl substituent is pyridyl, pyridazinyl, pyrimidinyl, pyrazinyl or triazinyl.

In an embodiment of the first aspect, R¹ is substituted by one or two groups independently selected from halo, C₁₋₆alkyl, C₁₋₆alkoxy, C₁₋₆haloalkyl, C₁₋₆haloalkoxy, cyano, C₁₋₆alkylthio, C₁₋₆haloalkylthio, C₁₋₆alkylsulfonyl, C₁₋₆haloalkylsulfonyl, C₁₋₆alkylcarbonyl and C₁₋₆haloalkylcarbonyl.

In an embodiment of the first aspect R¹ is phenyl or pyridyl either of which are optionally substituted by one or two groups independently selected from halo, C₁₋₆alkyl, C₁₋₆alkoxy, C₁₋₆haloalkyl, C₁₋₆haloalkoxy, C₁₋₆alkylthio and C₁₋₆haloalkylthio.

In an embodiment of the first aspect R² is C₁₋₆alkyl or C₁₋₄alkoxyC₁₋₄alkyl. In a further embodiment of the first aspect, R² is C₁₋₆alkyl.

In an embodiment, Y is O.

In an embodiment of the first aspect, A is 6-membered heteroaromatic ring; wherein X is CH or N; and wherein the ring may contain a total of 1 to 4 nitrogen atoms. In a further embodiment of the first aspect, A is 6-membered heteroaromatic ring; wherein X is CH or N; and wherein the ring contains a total of 1 or 2 nitrogen atoms. In a still further embodiment of the first aspect, A is a pyridine ring. In a still further embodiment of the first aspect, A is a pyridine ring attached to the amide nitrogen in formula (I) at the 4-position, i.e. having the following structure where the arrow indicates the point of attachment.

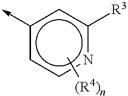

In an embodiment of the first aspect, R³ is halo, C₁₋₆alkyl, C₁₋₆haloalkyl or cyano.

In an embodiment of the first aspect, n is 0.

In an embodiment of the first aspect, the compound of formula (I) is selected from:
1-(4-fluorophenyl)-5-methyl-N-(6-methyl-2-pyridinyl)-1H-1,2,3-triazole-4-carboxamide (Compound 1);
1-(4-fluorophenyl)-5-methyl-N-(2-methyl-4-pyrimidinyl)-1H-1,2,3-triazole-4-carboxamide (Compound 2);
N-(3-chlorophenyl)-5-methyl-1-[3-(methylthio)phenyl]-1H-1,2,3-triazole-4-carboxamide (Compound 4);
N-(3-chlorophenyl)-1-(2,5-difluorophenyl)-5-methyl-1H-1,2,3-triazole-4-carboxamide (Compound 5):
N-(3-chlorophenyl)-5-methyl-1-phenyl-1H-1,2,3-triazole-4-carboxamide (Compound 6);
N-(3-chlorophenyl)-1-(4-fluorophenyl)-5-methyl-1H-1,2,3-triazole-4-carboxamide (Compound 7);
N-(3-fluorophenyl)-1-(4-fluorophenyl)-5-methyl-1H-1,2,3-triazole-4-carboxamide (Compound 8);
N-(3-chlorophenyl)-5-methyl-1-(2-methylphenyl)-1H-1,2,3-triazole-4-carboxamide (Compound 9);
N-(3-chlorophenyl)-1-(2-fluorophenyl)-5-methyl-1H-1,2,3-triazole-4-carboxamide (Compound 10);
1-(3-chloro-4-fluorophenyl)-N-(3-fluorophenyl)-5-methyl-1H-1,2,3-triazole-4-carboxamide (Compound 11);
N-(3-chlorophenyl)-1-(3-fluorophenyl)-5-methyl-1H-1,2,3-triazole-4-carboxamide (Compound 13);
N-(3-chlorophenyl)-1-(3,4-dimethylphenyl)-5-methyl-1H-1,2,3-triazole-4-carboxamide (Compound 14)
1-(4-fluorophenyl)-N-(6-fluoro-2-pyridinyl)-5-methyl-1H-1,2,3-triazole-4-carboxamide (Compound 20);
N-(6-chloro-2-pyridinyl)-1-(4-fluorophenyl)-5-methyl-1H-1,2,3-triazole-4-carboxamide (Compound 21);
1-(4-fluorophenyl)-5-methyl-N-(2-methyl-4-pyridinyl)-1H-1,2,3-triazole-4-carboxamide (Compound 22);
1-(3-chloro-4-fluorophenyl)-5-methyl-N-(2-methyl-4-pyrimidinyl)-1H-1,2,3-triazole-4-carboxamide (Compound 25);
N-(3-cyanophenyl)-1-(4-fluorophenyl)-5-methyl-1H-1,2,3-triazole-4-carboxamide (Compound 26);
1-(3,4-dimethylphenyl)-5-methyl-N-(2-methyl-4-pyrimidinyl)-1H-1,2,3-triazole-4-carboxamide (Compound 27);
1-(3-chloro-4-fluorophenyl)-5-methyl-N-(2-methyl-4-pyridinyl)-1H-1,2,3-triazole-4-carboxamide (Compound 28);
1-(3,4-difluorophenyl)-5-methyl-N-(2-methyl-4-pyridinyl)-1H-1,2,3-triazole-4-carboxamide (Compound 29); and
N-(3-chlorophenyl)-1-(5-fluoro-2-pyridinyl)-5-methyl-1H-1,2,3-triazole-4-carboxamide (Compound 31); or a pharmaceutically acceptable salt of any of the compounds.

In an embodiment of the first aspect, the compound of formula (I) is selected from:
1-(4-fluorophenyl)-5-methyl-N-(6-methyl-2-pyridinyl)-1H-1,2,3-triazole-4-carboxamide (Compound 1);
1-(4-fluorophenyl)-5-methyl-N-(2-methyl-4-pyrimidinyl)-1H-1,2,3-triazole-4-carboxamide (Compound 2);
1-(4-fluorophenyl)-N-(6-fluoro-2-pyridinyl)-5-methyl-1H-1,2,3-triazole-4-carboxamide (Compound 20);
N-(6-chloro-2-pyridinyl)-1-(4-fluorophenyl)-5-methyl-1H-1,2,3-triazole-4-carboxamide (Compound 21);
1-(4-fluorophenyl)-5-methyl-N-(2-methyl-4-pyridinyl)-1H-1,2,3-triazole-4-carboxamide (Compound 22);
1-(3-chloro-4-fluorophenyl)-5-methyl-N-(2-methyl-4-pyrimidinyl)-1H-1,2,3-triazole-4-carboxamide (Compound 25);
1-(3,4-dimethylphenyl)-5-methyl-N-(2-methyl-4-pyrimidinyl)-1H-1,2,3-triazole-4-carboxamide (Compound 27);
1-(3-chloro-4-fluorophenyl)-5-methyl-N-(2-methyl-4-pyridinyl)-1H-1,2,3-triazole-4-carboxamide (Compound 28); and
1-(3,4-difluorophenyl)-5-methyl-N-(2-methyl-4-pyridinyl)-1H-1,2,3-triazole-4-carboxamide (Compound 29); or a pharmaceutically acceptable salt of any of the compounds.

A number of structures defined in the first aspect have been disclosed previously. However none of these structures have been disclosed in association with any therapy.

According to a second aspect, the invention provides a compound of formula (I) or a salt thereof, wherein
R¹ is phenyl or 6-membered monocyclic heteroaryl, either of which are optionally substituted by one or two groups independently selected from halo, C₁₋₆alkyl, C₁₋₆alkoxy, C₁₋₆haloalkyl, C₁₋₆haloalkoxy, cyano, C₁₋₆alkylthio, C₁₋₆haloalkylthio, C₁₋₆alkylsulfonyl, C₁₋₆haloalkylsulfonyl, C₁₋₆alkylcarbonyl and C₁₋₆haloalkylcarbonyl;
R² is halo, C₁₋₆alkyl, C₁₋₆alkoxy, C₁₋₆haloalkyl, C₁₋₆haloalkoxy or C₁₋₄alkoxyC₁₋₄alkyl;
Y is O;
A is a 6-membered heteroaromatic ring; wherein X is CH, N, S or O; and wherein when A is a 5 or 6-membered heteroaromatic ring, the ring may contain a total of 1 to 4 heteroatoms independently selected from N, O and S;
R³ is halo, C₁₋₆alkyl, C₁₋₆alkoxy, C₁₋₆haloalkyl, C₁₋₆haloalkoxy, C₁₋₄alkoxyC₁₋₄alkyl or cyano;
R⁴ is halo; and
n is 0 or 1.

In an embodiment of the second aspect R¹ is phenyl or pyridyl either of which are optionally substituted by one or two groups independently selected from halo, C₁₋₆alkyl, C₁₋₆alkoxy, C₁₋₆haloalkyl, C₁₋₆haloalkoxy, C₁₋₆alkylthio and C₁₋₆haloalkylthio.

In an embodiment of the second aspect R² is C₁₋₆alkyl or C₁₋₄alkoxyC₁₋₄alkyl. In a further embodiment of the second aspect, R² is C₁₋₆alkyl.

In an embodiment of the second aspect, A is 6-membered heteroaromatic ring; wherein X is CH or N; and wherein the ring may contain a total of 1 to 4 nitrogen atoms. In a further embodiment of the first aspect, A is 6-membered heteroaromatic ring; wherein X is CH or N; and wherein the ring contains a total of 1 or 2 nitrogen atoms. In a still further embodiment of the second aspect, A is a pyridine ring. In a still further embodiment of the second aspect, A is a pyridine ring attached to the amide nitrogen in formula (I) at the 4-position, i.e. having the following structure where the arrow indicates the point of attachment.

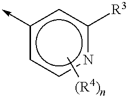

In an embodiment of the second aspect, R³ is halo, C₁₋₆alkyl, C₁₋₆haloalkyl or cyano.

In an embodiment of the second aspect, n is 0.

In an embodiment of the second aspect, the compound of formula (I) is selected from:
1-(4-fluorophenyl)-5-methyl-N-(6-methyl-2-pyridinyl)-1H-1,2,3-triazole-4-carboxamide (Compound 1);
1-(4-fluorophenyl)-5-methyl-N-(2-methyl-4-pyrimidinyl)-1H-1,2,3-triazole-4-carboxamide (Compound 2);
1-(4-fluorophenyl)-N-(6-fluoro-2-pyridinyl)-5-methyl-1H-1,2,3-triazole-4-carboxamide (Compound 20);
N-(6-chloro-2-pyridinyl)-1-(4-fluorophenyl)-5-methyl-1H-1,2,3-triazole-4-carboxamide (Compound 21);
1-(4-fluorophenyl)-5-methyl-N-(2-methyl-4-pyridinyl)-1H-1,2,3-triazole-4-carboxamide (Compound 22);
1-(3-chloro-4-fluorophenyl)-5-methyl-N-(2-methyl-4-pyrimidinyl)-1H-1,2,3-triazole-4-carboxamide (Compound 25);
1-(3,4-dimethylphenyl)-5-methyl-N-(2-methyl-4-pyrimidinyl)-1H-1,2,3-triazole-4-carboxamide (Compound 27);
1-(3-chloro-4-fluorophenyl)-5-methyl-N-(2-methyl-4-pyridinyl)-1H-1,2,3-triazole-4-carboxamide (Compound 28); and
1-(3,4-difluorophenyl)-5-methyl-N-(2-methyl-4-pyridinyl)-1H-1,2,3-triazole-4-carboxamide (Compound 29); or a salt of any of the compounds.

According to a third aspect, the invention provides a compound of formula (I) or a salt thereof, wherein
R¹ is a 6-membered monocyclic heteroaryl optionally substituted by one or two groups independently selected from halo, C₁₋₆alkyl, C₁₋₆alkoxy, C₁₋₆haloalkyl, C₁₋₆haloalkoxy, cyano, C₁₋₆alkylthio, C₁₋₆haloalkylthio, C₁₋₆alkylsulfonyl, C₁₋₆haloalkylsulfonyl, C₁₋₆alkylcarbonyl and C₁₋₆haloalkylcarbonyl;
R² is halo, C₁₋₆alkyl, C₁₋₆alkoxy, C₁₋₆haloalkyl, C₁₋₆haloalkoxy or C₁₋₄alkoxyC₁₋₄alkyl;
Y is O;
A is either a 6-membered heteroaromatic ring or a benzene ring; wherein X is CH, N, S or O; and wherein when A is a 6-membered heteroaromatic ring, the ring may contain a total of 1 to 4 heteroatoms independently selected from N, O and S;
R³ is halo, C₁₋₆alkyl, C₁₋₆alkoxy, C₁₋₆haloalkyl, C₁₋₆haloalkoxy, C₁₋₄alkoxyC₁₋₄alkyl or cyano;
R⁴ is halo; and
n is 0 or 1.

In an embodiment of the third aspect R¹ is pyridyl optionally substituted by one or two groups independently selected from halo, C₁₋₆alkyl, C₁₋₆alkoxy, C₁₋₆haloalkyl, C₁₋₆haloalkoxy, C₁₋₆alkylthio and C₁₋₆haloalkylthio.

In an embodiment of the third aspect R² is C₁₋₆alkyl or C₁₋₄alkoxyC₁₋₄alkyl. In a further embodiment of the third aspect, R² is C₁₋₆alkyl.

In an embodiment of the third aspect, A is 6-membered heteroaromatic ring; wherein X is CH or N; and wherein the ring may contain a total of 1 to 4 nitrogen atoms. In a further embodiment of the third aspect, A is 6-membered heteroaromatic ring; wherein X is CH or N; and wherein the ring contains a total of 1 or 2 nitrogen atoms. In a still further embodiment of the third aspect, A is a pyridine ring. In a still further embodiment of the third aspect, A is a pyridine ring attached to the amide nitrogen in formula (I) at the 4-position, i.e. having the following structure where the arrow indicates the point of attachment.

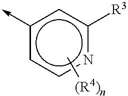

In an embodiment of the third aspect, R³ is halo, C₁₋₆alkyl, C₁₋₆haloalkyl or cyano.

In an embodiment of the third aspect, n is 0.

In an embodiment of the third aspect the compound of formula (I) is N-(3-chlorophenyl)-1-(5-fluoro-2-pyridinyl)-5-methyl-1H-1,2,3-triazole-4-carboxamide (Compound 31); or a salt thereof.

The compounds of formula (I) as defined in the first, second and third aspect may contain a basic centre and may form non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, Hl, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497. In an embodiment, the salt defined in the second and third aspects is pharmaceutically acceptable.

It will be appreciated by those skilled in the art that certain protected derivatives of the compounds of formula (I) as defined in the first, second and third aspects, which may be made prior to a final deprotection stage, may not possess pharmacological activity as such, but may, in certain instances, be administered orally or parenterally and thereafter metabolised in the body to form compounds defined in the first or second aspects which are pharmacologically active. Such derivatives may therefore be described as “prodrugs”. All protected derivatives and prodrugs of compounds defined in the first, second or third aspects are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compounds defined in the first, second or third aspects. Therefore, in a further aspect, the invention provides a prodrug of a compound defined in the first, second or third aspects.

The compounds of formula (I) defined in the first, second and third aspects, their salts or prodrugs, may exist in solvated or hydrated form. Therefore, in a further aspect, the invention provides a solvate or hydrate of a compound defined in the first or second aspects or a salt thereof.

The compounds of formula (I) defined in the first, second or third aspects, or their salts, or solvates or hydrates of either, may exist in one or more polymorphic form. Therefore, in a further aspect, the invention provides a polymorph of a) a compound of formula (I) or salt thereof defined in the first, second or third aspects or b) a solvate or hydrate of a compound of formula (I) or slat thereof defined in the first, second or third aspects.

Hereinafter, compounds of formula (I) as defined in the first, second and third aspects, their salts and prodrugs; any solvates or hydrates of any salt or prodrug; and any polymorph of any compound, salt, solvate or hydrate are referred to as “compounds of the invention”. The term “compounds of the invention” also includes all embodiments described for the first, second and third aspects.

The compounds of the invention may possess one or more chiral centres and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilising methods known to those skilled in the art. In addition, chiral intermediate compounds may be resolved and used to prepare chiral compounds of the invention. In addition, the chiral compounds of the invention may be prepared by chiral synthesis.

The compounds of the invention may exist in one or more tautomeric forms. All tautomers and mixtures thereof are included in the scope of the present invention. For example, a claim to 2-hydroxyquinolinyl would also cover its tautomeric form, α-quinolinonyl.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹¹C, ¹³C, ¹⁴C, ¹⁵N, ¹⁷O, ¹⁸O, ³¹P, ³²P, ³⁵S, ¹⁸F and ³⁶Cl, respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Experimental section hereafter using appropriate isotopic variations of suitable reagents.

Compounds of the invention may be prepared in a variety of ways. In the following reaction schemes and hereafter, unless otherwise stated R¹ to R⁴, X, Y, n etc are as defined in the first, second or third aspects. These processes form further aspects of the invention.

Throughout the specification, general formulae are designated by Roman numerals (I), (II), (III), (IV) etc. Subsets of these general formulae are defined as (Ia), (Ib), (Ic) etc . . . (IVa), (IVb), (IVc) etc.

Compounds of formula (Ia), i.e. compounds of general formula (I) where Y is O, may be prepared by reacting compounds of formula (II) with compounds of formula (III) according to reaction scheme 1, wherein R is alkyl. Typical reaction conditions comprise reacting (II) with (III) in the presence of AIMe₃ in THF with heating.

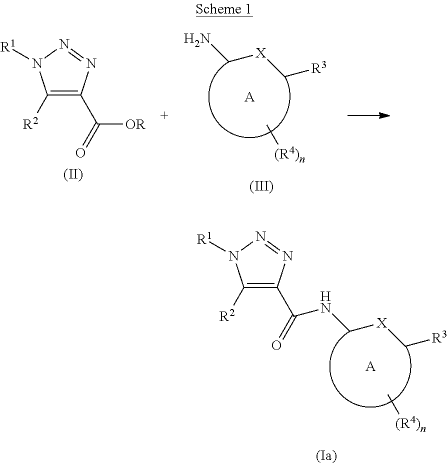

Compounds of formula (Ia), i.e. compounds of general formula (I) where Y is O, may also be prepared by reacting compounds of formula (IV) with compounds of formula (III) according to reaction scheme 2. Typical reaction conditions comprise reacting (IV) with (III) in the presence of HOAt/HATU, DIPEA and NMP at room temperature. Alternative reaction conditions comprise reacting (IV) with (III) in the presence of ethandioyl dichloride in DMF and N,N-dimethyl-4-pyridinamine in DCM at room temperature.

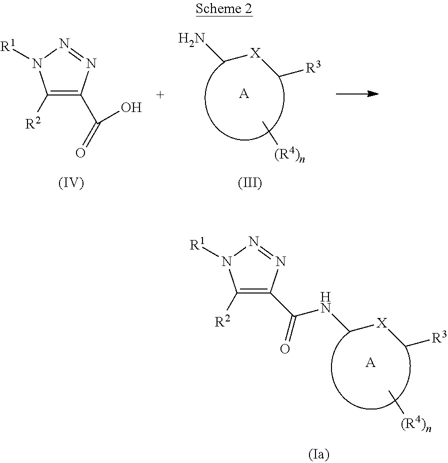

Compounds of formula (II) may be prepared by reacting compounds of formula (VI), wherein R is alkyl, with an azide of formula (V) according to reaction scheme 3. Typical reaction conditions comprise reacting (V) with (VI) in the presence of a trialkylamine in methanol with heating (see Tetrahedron, Vol. 44, No2, 1988, pp 461-469).

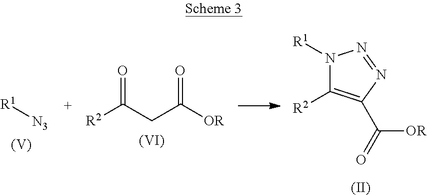

Alternatively compounds of formula (II) may be prepared by reacting compounds of formula (VII) with an azide of formula (V) according to reaction scheme 4. Typical reaction conditions comprise reacting (V) with (VII) in toluene and heating (see Tetraheadron, Vol. 44, No 2 1988, pp 461-469),

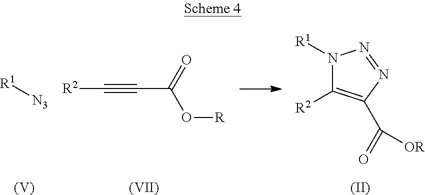

Alternatively, compounds of formula (II) may be prepared in two steps according to reaction scheme 5. Firstly compounds of formula (V) may be reacted with compounds of formula (VIII) to give compounds of formula (IV). Typical reaction conditions comprise reacting azide (V) with a Grignard reagent (VIII) in the presence of carbon dioxide in THF at room temperature to produce a compound of formula (IV) (see Organic Lett., 2006, Vol. 6 No 8 1237). The compound of formula (IV) may then be esterified to compounds of formula (II). Suitable reaction condition comprise heating (IV) with a suitable alcohol in the presence of sulfuric acid.

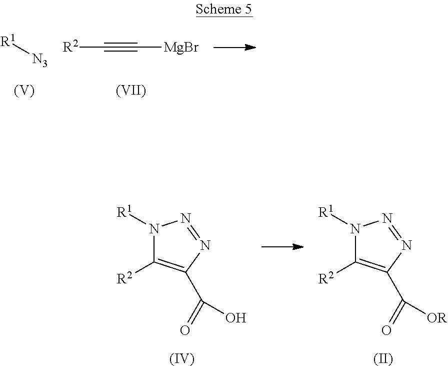

Compounds of formula (V) may be prepared according to procedures known to the skilled person. For example, see Organic. Lett., 2007, Vol. 9 No. 9 1809; Tet. Lett., 48 (2007) 3525. Compounds of formula (VIII) may also be prepared according to procedures known to the skilled person. For example, see Tetraheadron, Vol. 44, No 2 1988, pp 461-469.

Compounds of formula (Ib), i.e. compounds of general formula (I) where Y is S, may be prepared from compounds of formula (Ia) by treatment with Lawesson's reagent

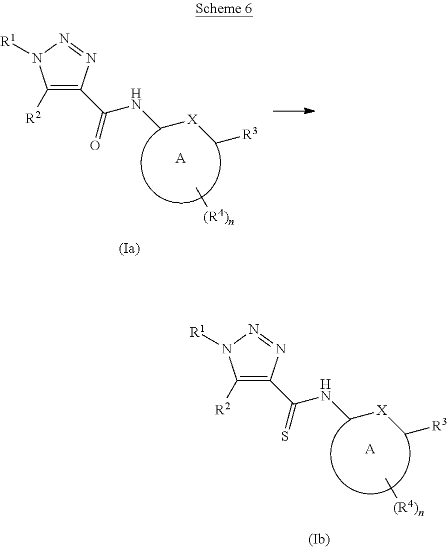

It will be appreciated by the skilled chemist that compounds of formula (I) may be converted to other compounds of formula (I) by methods known in the art.

The compounds of the invention antagonise the mGluR5 receptor and may be used to treat diseases or conditions for which antagonism of the mGluR5 receptor is beneficial. Therefore, in a further embodiment of the first aspect, the therapy is to treat a human disease or condition for which antagonism of the mGluR5 receptor is beneficial. In a still further embodiment of the first aspect, the therapy is selected from the list consisting of: [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Psychotic disorders for example Schizophrenia (including the subtypes Paranoid Type (295.30), Disorganised Type (295.10), Catatonic Type (295.20), Undifferentiated Type (295.90) and Residual Type (295.60)); Schizophreniform Disorder (295.40); Schizoaffective Disorder (295.70) (including the subtypes Bipolar Type and Depressive Type); Delusional Disorder (297.1) (including the subtypes Erotomanic Type, Grandiose Type, Jealous Type, Persecutory Type, Somatic Type, Mixed Type and Unspecified Type); Brief Psychotic Disorder (298.8); Shared Psychotic Disorder (297.3); Psychotic Disorder due to a General Medical Condition (including the subtypes with Delusions and with Hallucinations); Substance-Induced Psychotic Disorder (including the subtypes with Delusions (293.81) and with Hallucinations (293.82)); and Psychotic Disorder Not Otherwise Specified (298.9).
ii) Depression and mood disorders for example Depressive Episodes (including Major Depressive Episode, Manic Episode, Mixed Episode and Hypomanic Episode); Depressive Disorders (including Major Depressive Disorder, Dysthymic Disorder (300.4), Depressive Disorder Not Otherwise Specified (311)); Bipolar Disorders (including Bipolar I Disorder, Bipolar II Disorder (i.e. Recurrent Major Depressive Episodes with Hypomanic Episodes) (296.89), Cyclothymic Disorder (301.13) and Bipolar Disorder Not Otherwise Specified (296.80)); Other Mood Disorders (including Mood Disorder due to a General Medical Condition (293.83) which includes the subtypes With Depressive Features, With Major Depressive-like Episode, With Manic Features and With Mixed Features); Substance-Induced Mood Disorder (including the subtypes With Depressive Features, With Manic Features and With Mixed Features); and Mood Disorder Not Otherwise Specified (296.90).
iii) Anxiety disorders for example Social Anxiety Disorder; Panic Attack; Agoraphobia, Panic Disorder; Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder; and Anxiety Disorder Not Otherwise Specified (300.00).
iv) Substance-related disorders for example Substance Use Disorders (including Substance Dependence, Substance Craving and Substance Abuse); Substance-Induced Disorders (including Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders (including Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9)); Amphetamine (or Amphetamine-Like)-Related Disorders (for example Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9)); Caffeine Related Disorders (including Caffeine Intoxication (305.90), Caffeine-Induced Anxiety Disorder, Caffeine-Induced Sleep Disorder and Caffeine-Related Disorder Not Otherwise Specified (292.9)); Cannabis-Related Disorders (including Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9)); Cocaine-Related Disorders (including Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9)); Hallucinogen-Related Disorders (including Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9)); Inhalant-Related Disorders (including Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9)); Nicotine-Related Disorders (including Nicotine Dependence (305.1), Nicotine Withdrawal (292.0) and Nicotine-Related Disorder Not Otherwise Specified (292.9)); Opioid-Related Disorders (including Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9)); Phencyclidine (or Phencyclidine-Like)-Related Disorders (including Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9)); Sedative-, Hypnotic-, or Anxiolytic-Related Disorders (including Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9)); Polysubstance-Related Disorder (including Polysubstance Dependence (304.80)); and Other (or Unknown) Substance-Related Disorders (including Anabolic Steroids, Nitrate Inhalants and Nitrous Oxide).
v) Sleep disorders for example primary sleep disorders such as Dyssomnias (including Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47)); primary sleep disorders such as Parasomnias (including Nightmare Disorder (307.47), Sleep Terror Disorder (307.46), Sleepwalking Disorder (307.46) and Parasomnia Not Otherwise Specified (307.47)); Sleep Disorders Related to Another Mental Disorder (including Insomnia Related to Another Mental Disorder (307.42) and Hypersomnia Related to Another Mental Disorder (307.44)); Sleep Disorder Due to a General Medical Condition; and Substance-Induced Sleep Disorder (including the subtypes Insomnia Type, Hypersomnia Type, Parasomnia Type and Mixed Type).
vi) Eating disorders such as Anorexia Nervosa (307.1) (including the subtypes Restricting Type and Binge-Eating/Purging Type); Bulimia Nervosa (307.51) (including the subtypes Purging Type and Nonpurging Type); Obesity; Compulsive Eating Disorder; Binge Eating Disorder; and Eating Disorder Not Otherwise Specified (307.50).
vii) Autism Spectrum Disorders including Autistic Disorder (299.00), Asperger's Disorder, Rett's Disorder, Childhood Disintegrative Disorder and Pervasive Developmental Disorder Not Otherwise Specified.
viii) Attention-Deficit/Hyperactivity Disorder (including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9)); Hyperkinetic Disorder; Disruptive Behaviour Disorders such as Conduct Disorder (including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).
ix) Enhancement of cognition including the treatment of cognition impairment in other diseases such as schizophrenia, bipolar disorder, depression, other psychiatric disorders and psychotic conditions associated with cognitive impairment, e.g. Alzheimer's disease.

In an embodiment of the first aspect, the therapy is selected from the list consisting of: Parkinson's Disease, epilepsy, inflammatory pain, neuropathic pain, migraine, Down's Syndrome, gastroesophageal reflux disease.

In a further embodiment of the first aspect, the therapy is neuroprotection.

In a further embodiment of the first aspect, the therapy is a substance-related disorder.

It will be appreciated that references herein to “treat”, “treating” or “treatment” extend to prophylaxis, prevention of recurrence and suppression or amelioration of symptoms (whether mild, moderate or severe) as well as the treatment of established conditions.

It will be appreciated that the invention includes the following further aspects. The embodiments described above, where appropriate, extend to these further aspects.

i) A use of a compound of the invention in the manufacture of a medicament for treating a disease or condition for which antagonism of the mGluR5 receptor is beneficial. In an embodiment the disease or condition is a substance related disorder.
ii) A method of treating a disease or condition in a mammal for which antagonism of the mGluR5 receptor is beneficial comprises administering an effective amount of a compound of the invention. In an embodiment the disease or condition is a substance related disorder. In an embodiment the mammal is a human.

The compounds of the invention will normally, but not necessarily, be formulated into pharmaceutical compositions prior to administration to a patient by an appropriate route. Accordingly, in another aspect, the invention provides pharmaceutical compositions comprising a compound of the invention and one or more pharmaceutically-acceptable excipients.

As used herein, “pharmaceutically-acceptable excipient” means any pharmaceutically acceptable material present in the pharmaceutical composition or dosage form other than the compound or compounds of the invention. Typically the material gives form, consistency and performance to the pharmaceutical composition.

The pharmaceutical compositions of the invention typically contain one compound of the invention. However, in certain embodiments, the pharmaceutical compositions of the invention contain more than one compound of the invention. In addition, the pharmaceutical compositions of the invention may comprise one or more additional pharmaceutically active compounds.

Such pharmaceutical compositions of the invention may be prepared and packaged in bulk form wherein a safe and effective amount of a compound of the invention can be dispensed and then given to the patient such as with powders or syrups. Alternatively, the pharmaceutical compositions of the invention may be prepared and packaged as dosage forms wherein each physically discrete dosage form contains a safe and effective amount of a compound of the invention. Accordingly, in another aspect, the invention provides dosage forms comprising pharmaceutical compositions of the invention. Each discrete dosage form typically contains from 0.1 mg to 100 mg of a compound of the invention.

The compositions of the invention will typically be formulated into dosage forms which are adapted for administration to the patient by the desired route of administration. For example, dosage forms include those adapted for (1) oral administration such as tablets, capsules, caplets, pills, lozenges, powders, syrups, elixirs, suspensions, solutions, emulsions, sachets and cachets; (2) parenteral administration such as sterile solutions, suspensions, implants and powders for reconstitution; (3) transdermal administration such as transdermal patches; (4) rectal and vaginal administration such as suppositories, pessaries and foams; (5) inhalation and intranasal such as dry powders, aerosols, suspensions and solutions (sprays and drops); (6) topical administration such as creams, ointments, lotions, solutions, pastes, drops, sprays, foams and gels; (7) ocular administration such as drops, ointment, sprays, suspensions and inserts; (8) buccal and sublingual administration such as lozenges, patches, sprays, drops, chewing gums and tablets.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance. Certain pharmaceutically-acceptable excipients may be chosen for their ability to facilitate the release of the compound of the invention at the appropriate rate to treat the condition.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavouring agents, flavour masking agents, colouring agents, anticaking agents, humectants, chelating agents, plasticizers, viscosity increasing agents, rate modifying agents, antioxidants, preservatives, stabilizers, surfactants and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to determine suitable pharmaceutically-acceptable excipients in appropriate amounts for use with the compounds of the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press). The pharmaceutical compositions of the invention may be prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch and pre-gelatinized starch), gelatin, acacia, sodium alginate, alginic acid, tragacanth, guar gum, povidone, and cellulose and its derivatives (e.g. hydroxypropyl methyl cellulose). The oral solid dosage form may further comprise a disintegrant. Suitable disintegrants include starches, crospovidone, sodium starch glycolate, cros-carmellose, alginic acid, and sodium carboxymethyl cellulose. The oral solid dosage form may further comprise a lubricant. Suitable lubricants include stearic acid, magnesium stearate, calcium stearate, and sodium dodecyl sulphate. The oral solid dosage form may further comprise a glidant such as talc and colloidal silicon dioxide. The oral solid dosage form may further comprise an outer coating which may have cosmetic or functional properties.

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with antidepressants to treat or prevent depression and mood disorders.

The compounds of the invention may be used in combination with the following agents to treat or prevent bipolar disease: i) mood stabilisers; ii) antipsychotics; and iii) antidepressants.

The compounds of the invention may be used in combination with the following agents to treat or prevent anxiety disorders: i) anxiolytics; and ii) antidepressants.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine, sertraline femoxetine, fluvoxamine, indalpine and zimeldine); dual serotonin/noradrenaline reuptake inhibitors (such as venlafaxine, duloxetine and milnacipran); Noradrenaline reuptake inhibitors (such as reboxetine and venlafaxine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

It will be appreciated that the compounds of the combination or composition may be administered simultaneously (either in the same or different pharmaceutical formulations), separately or sequentially.

All publications, including, but not limited to, patents and patent applications cited in this specification, are herein incorporated by reference as if each individual publication were specifically and individually indicated to be incorporated by reference herein as though fully set forth.

### Supporting Compounds

The preparation of a number of compounds of the invention are described below.

In the procedures that follow, after each starting material, reference to an intermediate is typically provided. This is provided merely for assistance to the skilled chemist. The starting material may not necessarily have been prepared from the batch referred to.

Compounds of the invention and intermediates are named using ACD/Name PRO 6.02 chemical naming software (Advanced Chemistry Development Inc., Toronto, Ontario, M5H2L3, Canada).

### Abbreviations

Cmp—Compound
DCM—Dichloromethane
DIPEA—Diisopropylethylamine
DMSO—Dimethylsulphoxide
EtOAc—Ethylacetate
HATU—o-(7-azabenzotriazol-1-yl)-n,n,n′,n′-tetramethyluronium hexafluorophosphate
HOAt—1-hydroxy-7-azabenzotriazole
HPLC—High Performance Liquid Chromatography
MDAP—Mass Spec. Directed Preparative HPLC purification
NMP—N-methyl-2-pyrrolidone
NMR Nuclear Magnetic Resonance
TEA—Triethylamine

### Physical Characterisation

¹H NMR spectra were run on either a) a Bruker Avance 400 MHz instrument with a 5 mm QNP probe, b) a Bruker DPX-250 250 MHz instrument with a 5 mm QNP probe at 27 degC, or c) a Varian 300 MHz. Data were recorded in deuterio-chloroform, D6-dimethylsulphoxide or in D₄-deuterio-methanol with tetramethylsilane (TMS) as an internal reference. Chemical shifts given in parts per million (ppm) relative to TMS. Multiplicities are designated as s (singlet), d (doublet), t (triplet), dd (double-doublet), ddd (double-double-doublet), dt (double triplet), m (multiplet).

### Purification

A number of the compounds were purified using a Mass Directed Auto-Purification System (MDAP) incorporating HPLC techniques and an appropriate mass spectrometer such as the Waters® ZQ mass spectrometer.

Mass spectra (MS) were taken on a Agilent LC/MSD 1100 Mass Spectrometer, operating in ESI(+) ionization mode coupled with HPLC instrument Agilent 1100 Series [LC/MS-ESI(+): analysis performed on a Supelcosil ABZ+Plus (33×4.6 mm, 3 μm) (mobile phase: from 10%[CH₃CN+0.05% TFA] to 90%[CH₃CN+0.05% TFA] and 10% [water] in 2.2 min, under these conditions for 2.8 min. T=45° C., flux=0.9 mL/min).

### Intermediate 1: 6-Methyl-2-pyrazinamine

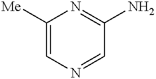

Dimethyl zinc (15.44 ml, 30.9 mmol) was added to a solution of 6-chloro-2-pyrazinamine (2 g, 15.44 mmol) and NiCl₂(dppp) (0.837 g, 1.544 mmol) in dry 1,4-dioxane (75 ml) and the mixture was refluxed for 24 hours. The mixture was then cooled to room temperature, quenched with methanol and concentrated in vacuum. The residue was partitioned between EtOAc and brine. The organic phase was dried over Na₂SO₄, filtered and the solvent evaporated. The residue was purified by silica chromatography (MeOH—NH₃-DCM) to afford the title compound as a pale yellow solid.

### Intermediate 2: Ethyl 1-(4-fluorophenyl)-5-methyl-1H-1,2,3-triazole-4-carboxylate

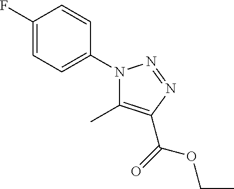

1-(4-Fluorophenyl)-5-methyl-1H-1,2,3-triazole-4-carboxylic acid [commercially available from ENAMINE Ltd. (23 Alexandra Matrosova Street, 01103 KIEV, Ukraine], 1 g, 4.52 mmol] was dissolved in ethanol (30 ml) and H₂SO₄ (0.254 ml, 4.52 mmol) was added at room temperature. The mixture was refluxed at 78° C. for 8 hours, cooled to room temperature and the solvent was evaporated. Water (50 ml) was added and the mixture was basified to pH=10 (NaOH, aq 2M) and extracted with EtOAc (3×50 ml). The organic extracts were combined and washed with an aqueous saturated solution of NaHCO₃ (2×50 ml). The organic extract was dried over Na₂SO₄, filtered and the solvent was evaporated to afford the title compound (680 mg); M+249.

### Intermediate 3: 5-Azido-2-fluoropyridine

### Method A

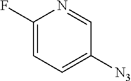

Sodium azide (0.430 g, 6.61 mmol) and copper(II) sulfate pentahydrate (0.138 g, 0.551 mmol) were placed in a round bottomed flask. Methanol (10 ml) and (6-fluoro-3-pyridinyl)boronic acid (0.776 g, 5.51 mmol) were added at room temperature and the mixture was stirred vigorously at room temperature for 3 days, until no more boronic acid could be detected by mass spectroscopy. The solvent was evaporated and petroleum ether was added. The mixture was stirred at room temperature for 10 min and then filtered through a pad of celite. Concentration of the filtrate gave the title compound as a brown oil (350 mg); ¹HNMR (d⁶ DMSO, 400 MHz) δ ppm: 7.26 (1H, dd), 7.83 (1H, m), 8.06 (1H, m).

### Method B

6-Fluoro-3-pyridinamine (2 g, 17.84 mmol) was dissolved in acetonitrile (40 ml) and the mixture was cooled to 0° C. 1,1-Dimethylethyl nitrite (3.18 ml, 26.8 mmol) was added at 0° C. followed by trimethylsilyl azide (2.82 ml, 21.41 mmol) and the mixture was stirred at room temperature for 5 hours. The solvent was evaporated and the residue was purified by silica gel chromatography (EtOAc-heptane) to afford the title compound (72.1%); ¹HNMR (DMSO, 400 MHz): 7.26 (1H dd), 7.83 (1H, m), 8.06 (1H, m).

### Intermediate 4: Ethyl 1-(3,5-difluorophenyl)-5-methyl-1H-1,2,3-triazole-4-carboxylate

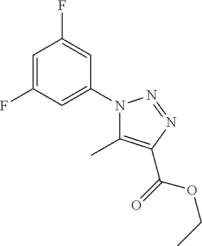

To a mixture of 1,3-difluoro-5-iodobenzene (0.2 g, 0.833 mmol) and ethyl 2-butynoate (0.097 ml, 0.833 mmol) in water/DMSO (0.22 ml/2 ml) at room temperature was added L-proline (0.019 g, 0.167 mmol), sodium carbonate (0.018 g, 0.167 mmol), sodium azide (0.065 g, 1.000 mmol), sodium 1-ascorbate (0.017 g, 0.083 mmol) and copper(II) sulfate pentahydrate (10.40 mg, 0.042 mmol) (in this order) and the mixture was heated at 65° C. overnight. Monitoring by LC/MS revealed starting material to be present and so the temperature was increased to 100 degrees and the mixture was heated overnight at 100 degC. On cooling to room temperature, the mixture was quenched with aqueous ammonia (1 ml) and EtOAc (20 ml) and water (20 ml) were added. The two phases were separated, and the aqueous phase was extracted with EtOAc (3×30 ml). All organic phases were combined and washed with water/brine (50 ml) repeatedly. The organic phase was dried over sodium sulfate, filtered and the solvent was evaporated to give a crude product that was purified by MDAP to afford the title compound (14.9%); MH+=267.9.

### Intermediate 5: Ethyl 1-(3-chloro-4-fluorophenyl)-5-methyl-1H-1,2,3-triazole-4-carboxylate

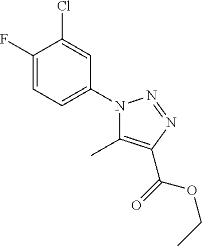

To a mixture of 2-chloro-1-fluoro-4-iodobenzene (0.5 g, 1.950 mmol) and ethyl 2-butynoate (0.227 ml, 1.950 mmol) in water/DMSO (0.22 ml/2 ml) at room temperature was added L-proline (0.045 g, 0.390 mmol), sodium carbonate (0.041 g, 0.390 mmol), sodium azide (0.152 g, 2.340 mmol), sodium 1-ascorbate (0.039 g, 0.195 mmol) and copper(II) sulfate pentahydrate (0.024 g, 0.097 mmol) (in this order) and the mixture was heated at 65° C. overnight. On cooling to room temperature, the mixture was quenched with aqueous ammonia (1 ml) and EtOAc (20 ml) and water (20 ml) were added. The two phases were separated, and the aqueous phase was extracted with EtOAc (3×30 ml). All organic phases were combined and washed with water/brine (50 ml) repeatedly. The organic phase was dried over sodium sulfate, filtered and the solvent was evaporated to afford a crude product that was purified by MDAP to afford the title compound (6.5%); MH+=284.

### Alternative Preparation:

3-Chloro-4-fluorophenyl azide (Intermediate 12) (618 mg, 3.60 mmol) was dissolved in toluene (6 ml). Ethyl 2-butynoate (0.420 ml, 3.60 mmol) was added and the mixture was stirred at 120° C. for 2 days. On cooling the mixture was purified by silica gel chromatography (40M, Biotage SP1) eluting with DCM to afford a mixture of the title compound (450 mg, 1.586 mmol, 44.0% yield) and its isomer ethyl 1-(3-chloro-4-fluorophenyl)-4-methyl-1H-1,2,3-triazole-5-carboxylate (373 mg, 1.315 mmol, 36.5% A yield); ¹H NMR (500 MHz, acetone) δ ppm 1.38 (t, J=7.12 Hz, 3H) 2.61 (s, 3H) 4.39 (q, J=7.09 Hz, 2H) 7.64 (t, J=8.80 Hz, 1H) 7.69-7.73 (m, 1H) 7.90 (dd, J=6.45, 2.48 Hz, 1H); UPLC-MS: 0.82 mins; [MH+/−]=284.

### Intermediate 6: Ethyl 1-(3,4-dimethylphenyl)-5-methyl-1H-1,2,3-triazole-4-carboxylate

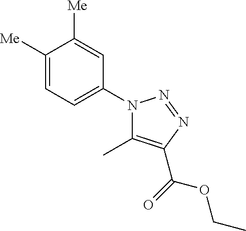

### Batch A

To a mixture of 4-iodo-1,2-dimethylbenzene (0.5 g, 2.155 mmol) and ethyl 2-butynoate (0.250 ml, 2.155 mmol) in water (0.22 ml)/DMSO (2 ml) at room temperature were added sodium 1-ascorbate (0.043 g, 0.215 mmol), L-proline (0.050 g, 0.431 mmol), sodium carbonate (0.046 g, 0.431 mmol), copper(II) sulfate pentahydrate (0.027 g, 0.108 mmol) and sodium azide (0.168 g, 2.59 mmol) (at the same time) and the mixture was heated at 65° C. for 48 hours. On cooling to room temperature, the mixture was quenched with aqueous ammonia (1 ml) and EtOAc (20 ml) and water (20 ml) were added. The two phases were separated, and the aqueous phase was extracted with EtOAc (3×30 ml). All organic phases were combined and washed with water/brine (50 ml) repeatedly. The organic phases were dried over sodium sulfate, filtered and the solvent was evaporated to afford a crude product that was purified by chromatography (25S silica column, 50/50 ethyl acetate/isohexane) to afford 7 mgs of impure product containing the title compound; MH+=260.

### Batch B

To a mixture of 4-iodo-1,2-dimethylbenzene (0.1 g, 0.431 mmol) and ethyl 2-butynoate (0.050 ml, 0.431 mmol) in water/DMSO (33 μl/0.3 ml) at room temperature were added sodium 1-ascorbate (8.54 mg, 0.043 mmol), L-proline (9.92 mg, 0.086 mmol), sodium carbonate (9.13 mg, 0.086 mmol), copper (II) sulfate pentahydrate (5.38 mg, 0.022 mmol), sodium azide (0.034 g, 0.517 mmol) (at the same time) and the mixture was heated in the microwave reactor at 100° C. for 40 min and then at 120° C. for 1 h. The mixture was quenched with aqueous ammonia (1 ml) and EtOAc (10 ml) and water (10 ml) were added. The two phases were separated, and the aqueous phase was extracted with EtOAc (3×15 ml). All organic phases were combined and washed with water/brine (20 ml) repeatedly. The organic phases were dried over Na₂SO₄, filtered and the solvent was evaporated to afford the crude product that was purified by MDAP to afford 6 mgs of impure product containing the title compound; MH+=260.

### Intermediate 7: Ethyl 1-(3,4-difluorophenyl)-5-methyl-1H-1,2,3-triazole-4-carboxylate

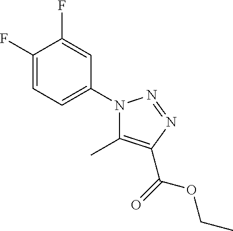

To a mixture of 1,2-difluoro-4-iodobenzene (1 g, 4.17 mmol) and ethyl 2-butynoate (0.484 mL, 4.17 mmol) in water (0.444 ml)/DMSO (4 ml) at room temperature were added L-proline (0.096 g, 0.833 mmol), sodium carbonate (0.088 g, 0.833 mmol), sodium azide (0.325 g, 5.00 mmol), sodium 1-ascorbate (0.083 g, 0.417 mmol) and copper(II) sulfate pentahydrate (0.052 g, 0.208 mmol) (in this order) and the mixture was heated at 65° C. overnight. On cooling to room temperature, the mixture was quenched with aqueous ammonia (1 ml) and EtOAc (20 ml) and water (20 ml) were added. The two phases were separated and the aqueous phase was extracted with EtOAc (3×30 ml). The organic phases were combined and washed with water/brine (50 ml) repeatedly. The organic phases were dried over sodium sulfate, filtered and the solvent was evaporated to afford a crude product that was purified by MDAP to afford the title compound (23.9%); MH+=267.9; ¹HNMR (DMSO, 400 MHz): 1.34 (3H t), 4.36 (2H, q), 7.57 (1H m), 7.76 (1H m), 7.94 (1H m).

### Intermediate 8: Methyl 1-(4-fluorophenyl)-5-methyl-1H-1,2,3-triazole-4-carboxylate

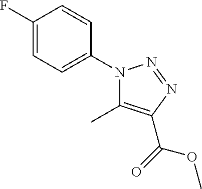

To a solution of 1-(4-fluorophenyl)-5-methyl-1H-1,2,3-triazole-4-carboxylic acid [commercially available from ENAMINE Ltd. (23 Alexandra Matrosova Street, 01103 KIEV, Ukraine] (200 mg, 0.95 mmol) in methanol (10 ml) was added concentrated H₂SO₄ (5 mg) and the mixture was heated under reflux overnight. The mixture was cooled, water was added (20 ml) and the mixture was extracted with ethyl acetate (3×20 ml). The combined organic phases were dried over sodium sulphate and the solvent removed to give the title compound (200 mg, 93.9%); MH+=236.1.

### Intermediate 9: Ethyl 1-(6-fluoro-3-pyridinyl)-5-methyl-1H-1,2,3-triazole-4-carboxylate

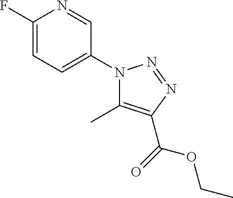

Ethyl magnesium chloride (1 ml, 2.000 mmol; 2M in THF) was added to an oven-dry flask and propyne (0.801 g, 20.00 mmol) was bubbled through the solution at 55° C. for 15 min. Dry THF (1 ml) was added and the mixture was cooled to room temperature. 5-Azido-2-fluoropyridine (0.276 g, 2.000 mmol) (Intermediate 9) in dry THF (0.5 ml) was added at room temperature and the mixture was stirred at 50 degrees for one hour. The mixture was cooled to room temperature and carbon dioxide (0.880 g, 20.00 mmol) was bubbled through for 10 min at room temperature whilst stirring. All these operations were performed under argon. The mixture was then diluted with water and acidified with aq. 5M HCl. EtOAc (50 ml) was added and the mixture was stirred vigorously. The two phases were separated and the aqueous phase was extracted with EtOAc (50 ml). All organic phases were combined, and the solvent was evaporated to a residue. The residue was partially dissolved in dichloromethane and filtered. The solid was collected and triturated with dichloromethane to afford the title compound (33.2%); MH+=223.

### Intermediate 10: Ethyl 1-(5-fluoro-2-pyridinyl)-5-methyl-1H-1,2,3-triazole-4-carboxylate

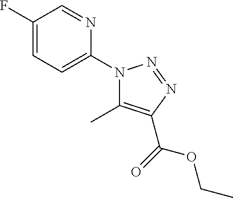

To a mixture of 2-bromo-5-fluoropyridine (0.5 g, 2.84 mmol) and ethyl 2-butynoate (0.319 g, 2.84 mmol) in water/DMSO (0.7 ml/6 ml) was added at room temperature L-proline (0.065 g, 0.568 mmol), sodium carbonate (0.060 g, 0.568 mmol), sodium azide (0.222 g, 3.41 mmol), sodium 1-ascorbate (0.056 g, 0.284 mmol) and copper(II) sulfate pentahydrate (0.035 g, 0.142 mmol) (in this order) and the mixture was heated at 65° C. overnight. Monitoring by LC/MS revealed starting material to be present and so the temperature was increased to 100 degrees and the mixture was heated overnight at 100 degC. The mixture was then quenched with aqueous ammonia (1 ml) and EtOAc (20 ml) and water (20 ml) were added. The two phases were separated, and the aqueous phase was extracted with EtOAc (3×30 ml). The combined organic phases were combined and washed with water/brine (50 ml) repeatedly. The organic phases were dried over sodium sulphate, filtered and the solvent was evaporated to afford a residue that was purified by MDAP to give the title compound (1.3%); ¹HNMR (DMSO, 400 MHz): 1.34 (3H t), 2.71 (3H s), 4.36 (2H, q), 8.03 (1H m), 8.15 (1H m), 8.73 (1H d).

### Intermediate 11: Ethyl 5-methyl-1-(6-methyl-3-pyridinyl)-1H-1,2,3-triazole-4-carboxylate

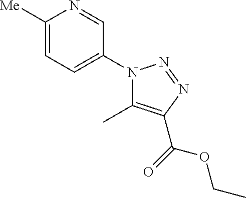

To a mixture of 5-iodo-2-methylpyridine (160 mg, 0.731 mmol) and ethyl 2-butynoate (0.085 ml, 0.731 mmol) in water/DMSO (0.2 ml/2 ml) was added at room temperature L-proline (16.82 mg, 0.146 mmol), sodium carbonate (15.49 mg, 0.146 mmol), sodium azide (57.0 mg, 0.877 mmol), sodium 1-ascorbate (14.47 mg, 0.073 mmol) and copper(II) sulfate pentahydrate (9.12 mg, 0.037 mmol) (in this order) and the mixture was heated at 65° C. overnight. Monitoring by LC/MS revealed starting material to be present and so the temperature was increased to 100 degrees and the mixture was heated overnight at 100 degC. The mixture was then quenched with aqueous ammonia (1 ml) and EtOAc (20 ml) and water (20 ml) were added. The two phases were separated, and the aqueous phase was extracted with EtOAc (3×30 ml). The organic phases were combined and washed with water/brine (50 ml) repeatedly. The organic phases were dried over sodium sulfate, filtered and the solvent was evaporated to afford a crude product that was purified by MDAP to give the title compound (6.7%) MH+=247.

### Intermediate 12: 3-Chloro-4-fluorophenyl azide

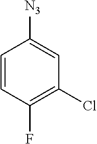

3-Chloro-4-fluoroaniline (2 g, 13.74 mmol) was dissolved in acetonitrile (40 mL). The solution was cooled to 0° C. and 1,1-dimethylethyl nitrite (2.451 mL, 20.61 mmol) was added followed by trimethylsilyl azide (2.168 mL, 16.49 mmol) and the mixture was left stirring while the temperature slowly rose to rt and left stirring at rt overnight. The solvent was evaporated (rotary, 25° C.) and the residue was purified by silica chromatography (25+M on Biotage SP1), eluting with Cyclohex:EtOAc 100:0 to 9:1. Product fractions were combined and the solvent removed to afford the title compound as a pale yellow oil (1.976 g, 11.52 mmol, 84% yield); ¹H NMR (400 MHz, CHLOROFORM-d): δ ppm 6.92 (ddd, J=8.83, 3.87, 2.78 Hz, 1H) 7.08-7.18 (m, 2H); UPLC-MS: 0.8 min, [MH+/−]=172.

### Compound 1: 1-(4-Fluorophenyl)-5-methyl-N-(6-methyl-2-pyridinyl)-1H-1,2,3-triazole-4-carboxamide

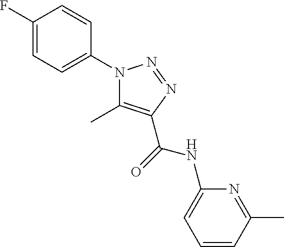

1-(4-Fluorophenyl)-5-methyl-1H-1,2,3-triazole-4-carboxylic acid (Commercially available from Enamine Ltd., 25 mg, 0.113 mmol) and 6-methyl-2-pyridinamine (12.22 mg, 0.113 mmol) in NMP (2 ml) and DIPEA (Hunig Base) (0.099 ml, 0.565 mmol) were stirred at room temperature. HOAt (30.8 mg, 0.226 mmol) and HATU (86 mg, 0.226 mmol) were added and the mixture was stirred at room temperature overnight. The mixture was partitioned between ethyl acetate and saturated sodium bicarbonate solution. The organic layer was separated, washed with water and brine (×2), dried (MgSO₄), filtered and concentrated under reduced pressure. The crude was partially dissolved in MeOH and filtered to afford the title compound (4 mgs); MH+312; ¹HNMR (d⁶ DMSO, 400 MHz) δ ppm: 2.45 (3H, s), 2.58 (3H, s), 7.07 (1H, d), 7.53 (2H, t), 7.77 (3H, m), 8.00 (1H, d), 9.73 (1H, s).

### Compound 2: 1-(4-Fluorophenyl)-5-methyl-N-(2-methyl-4-pyrimidinyl)-1H-1,2,3-triazole-4-carboxamide

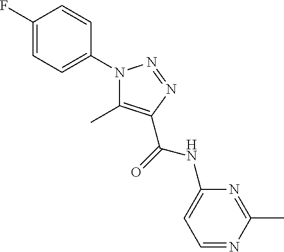

To a solution of 2-methyl-4-pyrimidinamine (0.180 g, 1.645 mmol) in dry 1,4-dioxane (2 ml) was added trimethylaluminum (0.802 ml, 1.605 mmol) dropwise and the solution was stirred at room temperature for 30 min. A solution of ethyl 1-(4-fluorophenyl)-5-methyl-1H-1,2,3-triazole-4-carboxylate (Intermediate 2, 0.1 g, 0.401 mmol) in dry dioxane (2 ml) was added at room temperature and the reaction was heated at 100° C. for 1.5 hours. The reaction was cooled to room temperature, quenched cautiously with water (0.125 ml) and then stirred at room temperature for 10 min. Sodium sulphate (0.5 g) was added and the mixture was stirred vigorously for 5 min. DCM was added and the mixture was stirred for an additional 10 min at room temperature. The solution was filtered through hydrophobic filters and the solvent was evaporated. The resulting residue was purified by MDAP to give the title compound (30 mg); MH+313; ¹HNMR (d⁶ DMSO, 400 MHz) δ ppm: 2.57 (6H, s), 7.53 (2H, m), 7.76 (2H, m), 8.00 (1H, d), 8.65 (1H, d), 10.2 (1H, s).

### Compound 3: 1-(4-Fluorophenyl)-5-methyl-N-(6-methyl-2-pyrazinyl)-1H-1,2,3-triazole-4-carboxamide

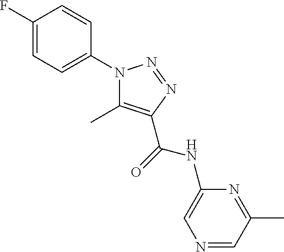

To a solution of 6-methyl-2-pyrazinamine (Intermediate 1, 0.180 g, 1.645 mmol) in dry 1,4-dioxane (2 ml) was added trimethylaluminium (0.802 ml, 1.605 mmol) dropwise and the solution was stirred at room temperature for 30 min. A solution of ethyl 1-(4-fluorophenyl)-5-methyl-1H-1,2,3-triazole-4-carboxylate (Intermediate 2, 0.1 g, 0.401 mmol) in dry dioxane (2 ml) was added at room temperature and the reaction was heated to 100° C. for 1.5 hours. The reaction was cooled to room temperature, quenched cautiously with water (0.125 ml) and stirred at room temperature for 10 min. Sodium sulphate (0.5 g) was added and the mixture was stirred vigorously for 5 min. Dichloromethane was added and the mixture was stirred for additional 10 min at room temperature. The solution was then filtered through hydrophobic filters, and the solvent was evaporated to afford the crude product that was purified by MDAP to give the title compound as a white solid; MH+313; ¹HNMR (CDCl₃, 400 MHz) δ ppm: 2.53 (3H, s), 2.69 (3H, s), 7.30 (2H, m), 7.49 (2H, m), 8.27 (1H, s), 9.47 (1H, s), 9.56 (1H, s br).

The following compounds (see Table 1) are commercially available and support the first aspect of the invention. The compounds are commercially available from at least one of the following commercial suppliers as indicated in the table: a) Life Chemicals Inc. (2477 Glenwood School Drive Suite 203, Burlington, ON, L7R 3R9, Canada); and b) Specs (Head Office, Kluyverweg 6, 2629 HT Delft, The Netherlands)

TABLE 1

| Cmp | Structure | Name |
|---|---|---|
| 4 | 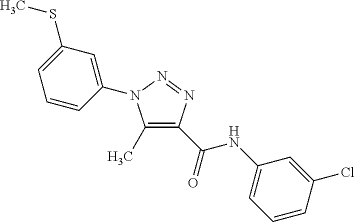 | N-(3-chlorophenyl)-5-methyl-1- [3-(methylthio)phenyl]-1H-1,2,3- triazole-4-carboxamide; Life Chemicals Inc. |
| 5 | 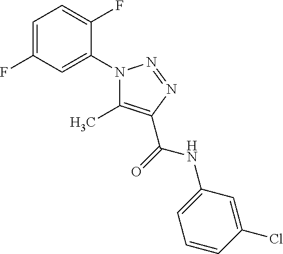 | N-(3-chlorophenyl)-1-(2,5- difluorophenyl)-5-methyl-1H- 1,2,3-triazole-4-carboxamide; Life Chemicals Inc. |
| 6 | 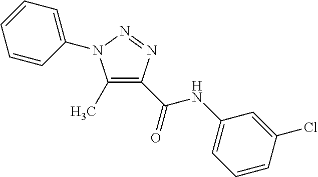 | N-(3-chlorophenyl)-5-methyl-1- phenyl-1H-1,2,3-triazole-4- carboxamide; Life Chemicals Inc. |
| 7 | 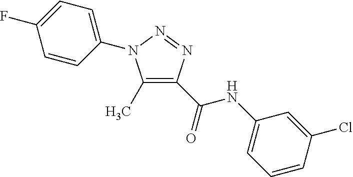 | N-(3-chlorophenyl)-1-(4- fluorophenyl)-5-methyl-1H-1,2,3- triazole-4-carboxamide; Life Chemicals Inc. |
| 8 | 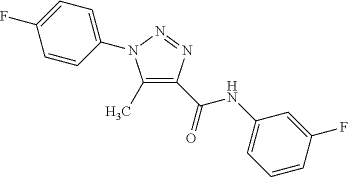 | N-(3-fluorophenyl)-1-(4- fluorophenyl)-5-methyl-1H-1,2,3- triazole-4-carboxamide; Life Chemicals Inc. |
| 9 | 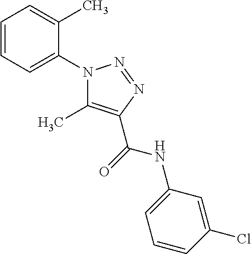 | N-(3-chlorophenyl)-5-methyl-1- (2-methylphenyl)-1H-1,2,3- triazole-4-carboxamide; Life Chemicals Inc. |
| 10 | 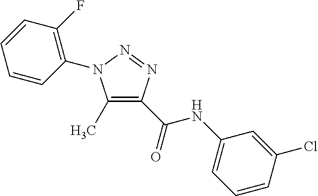 | N-(3-chlorophenyl)-1-(2- fluorophenyl)-5-methyl-1H-1,2,3- triazole-4-carboxamide; Life Chemicals Inc. |
| 11 | 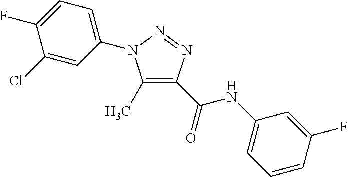 | 1-(3-chloro-4-fluorophenyl)-N-(3- fluorophenyl)-5-methyl-1 H-1,2,3- triazole-4-carboxamide; Life Chemicals Inc. |
| 12 | 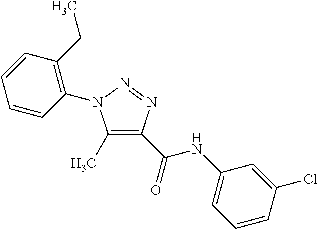 | N-(3-chlorophenyl)-1-(2- ethylphenyl)-5-methyl-1H-1,2,3- triazole-4-carboxamide; Life Chemicals Inc. |
| 13 | 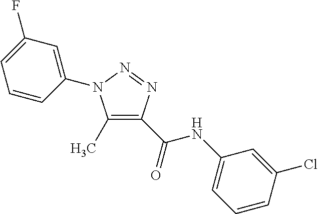 | N-(3-chlorophenyl)-1-(3- fluorophenyl)-5-methyl-1H-1,2,3- triazole-4-carboxamide; Life Chemicals Inc. |
| 14 | 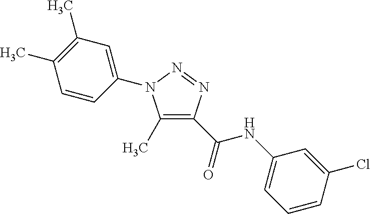 | N-(3-chlorophenyl)-1-(3,4- dimethylphenyl)-5-methyl-1H- 1,2,3-triazole-4-carboxamide; Life Chemicals Inc. |
| 15 | 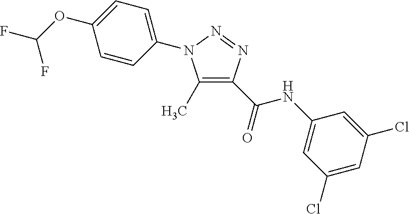 | N-(3,5-dichlorophenyl)-1-{4- [(difluoromethyl)oxy]phenyl}-5- methyl-1H-1,2,3-triazole-4- carboxamide; Specs |
| 16 | 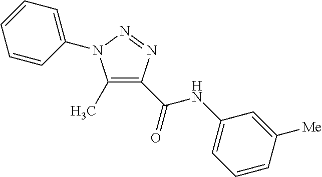 | 5-methyl-N-(3-methylphenyl)-1- phenyl-1H-1,2,3-triazole-4- carboxamide; Specs |
| 17 | 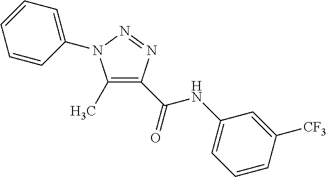 | 5-methyl-1-phenyl-N-[3- (trifluoromethyl)phenyl]-1H- 1,2,3-triazole-4-carboxamide; Specs |
| 18 | 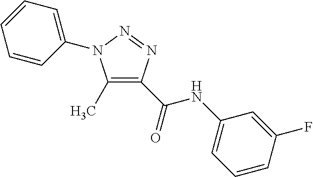 | N-(3-fluorophenyl)-5-methyl-1- phenyl-1H-1,2,3-triazole-4- carboxamide; Life Chemicals Inc. |

### Compound 19: 1-(4-Fluorophenyl)-5-methyl-N-[6-(trifluoromethyl)-2-pyridinyl]-1H-1,2,3-triazole-4-carboxamide

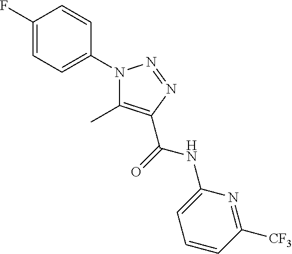

To a solution of 6-(trifluoromethyl)-2-pyridinamine (0.267 g, 1.645 mmol) in dry 1,4-dioxane (2 ml) was added dropwise trimethylaluminium (2M solution in toluene) (0.802 ml, 1.605 mmol). The solution was stirred at room temperature for 30 min then a solution of ethyl 1-(4-fluorophenyl)-5-methyl-1H-1,2,3-triazole-4-carboxylate (0.1 g, 0.401 mmol) (Intermediate 2) in dry dioxane (2 ml) was added at room temperature and the reaction was heated to 100° C. for 1.5 hours. The reaction was cooled to room temperature quenched cautiously with water (0.2 ml) and stirred at room temperature for 10 min. The mixture was then diluted with dichloromethane, stirred for 10 min, filtered and the solvent was evaporated to afford a residue that was left under vacuum for 12 hours. The residue was then treated with methanol (5 ml) and filtered to afford the title compound 35%, MH+=366; ¹HNMR (DMSO, 400 MHz): 2.60 (3H s), 7.53 (2H m), 7.70 (1H d), 7.77 (2H m), 8.18 (1H t), 8.45 (1H d), 10.38 (1H s).

### Compound 20:1-(4-Fluorophenyl)-N-(6-fluoro-2-pyridinyl)-5-methyl-1H-1,2,3-triazole-4-carboxamide

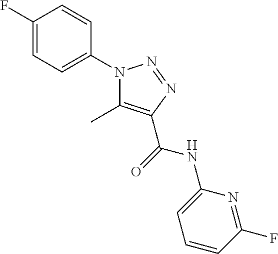

To a solution of 6-fluoro-2-pyridinamine (0.092 g, 0.822 mmol) in dry 1,4-dioxane (1 ml) was added dropwise trimethylaluminium (0.401 ml, 0.802 mmol) (2M solution in toluene). The solution was stirred at room temperature for 30 min. Then a solution of ethyl 1-(4-fluorophenyl)-5-methyl-1H-1,2,3-triazole-4-carboxylate (Intermediate 2) (0.05 g, 0.201 mmol) in dry 1,4-dioxane (1 ml) was added at room temperature and the reaction was heated to 100° C. for 1.5 hours. The reaction was cooled to room temperature, quenched cautiously with water (0.06 ml), and stirred at room temperature for 10 min. Then, sodium sulphate (0.25 g) was added to bind the water and the mixture was stirred vigorously for 5 min. Dichloromethane was added and the mixture was stirred for an additional 10 min at room temperature. The solution was filtered and the solid was washed with dichloromethane. The filtrate was evaporated to afford a residue. The residue was partially dissolved in methanol, filtered and the solid washed with diethyl ether to afford a the title compound (44.5%); MH+=316; ¹HNMR (DMSO, 400 MHz): 2.57 (3H s), 6.96 (1H m), 7.53 (2H t), 7.76 (2H m), 8.07 (2H m), 10.22 (1H s).

### Compound 21: N-(6-chloro-2-pyridinyl)-1-(4-fluorophenyl)-5-methyl-1H-1,2,3-triazole-4-carboxamide

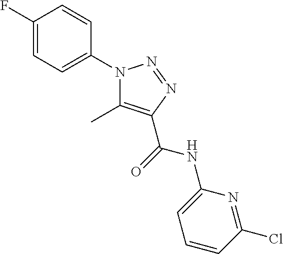

To a solution of 6-chloro-2-pyridinamine (0.106 g, 0.822 mmol) in dry 1,4-dioxane (1 ml) was added dropwise trimethylaluminium (0.401 ml, 0.802 mmol) (2M solution in toluene). The solution was stirred at room temperature for 30 min. Then a solution of ethyl 1-(4-fluorophenyl)-5-methyl-1H-1,2,3-triazole-4-carboxylate (0.05 g, 0.201 mmol) (Intermediate 2) in dry 1,4-dioxane (1 ml) was added at room temperature and the reaction was heated to 100° C. for 1.5 hours. The reaction was cooled to room temperature, quenched cautiously with water (0.06 ml), and stirred at room temperature for 10 min. Then, sodium sulphate (0.25 g) was added to bind the water and the mixture was stirred vigorously for 5 min. Dichloromethane was added and the mixture was stirred for an additional 10 min at room temperature. The solution was filtered and the solid was washed with dichloromethane. The filtrate was evaporated to afford a residue which was partially dissolved into methanol. The solution was filtered and the solid washed with diethyl ether to afford the title compound (10.9%); MH+=332; ¹HNMR (DMSO, 250 MHz): 7.31 (1H d), 7.53 (2H t), 7.76 (2H m), 7.94 (1H t), 8.15 (1H d), 10.28 (1H s).

### Compound 22: 1-(4-Fluorophenyl)-5-methyl-N-(2-methyl-4-pyridinyl)-1H-1,2,3-triazole-4-carboxamide

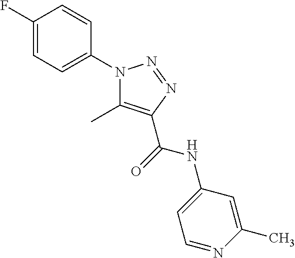

To a solution of 2-methyl-4-pyridinamine (0.089 g, 0.822 mmol) in dry 1,4-dioxane (1 ml) was added dropwise trimethylaluminium (0.401 ml, 0.802 mmol) (2M solution in toluene). The solution was stirred at room temperature for 30 min. Then a solution of ethyl 1-(4-fluorophenyl)-5-methyl-1H-1,2,3-triazole-4-carboxylate (0.05 g, 0.201 mmol) (Intermediate 2) in dry 1,4-dioxane (1 ml) was added at room temperature and the reaction was heated to 100° C. for 1.5 hours. The reaction was cooled to room temperature, quenched cautiously with water (0.06 ml), and stirred at room temperature for 10 min. Then, sodium sulphate (0.25 g) was added to bind the water and the mixture was stirred vigorously for 5 min. Dichloromethane was added and the mixture was stirred for an additional 10 min at room temperature. The solution was filtered and the solid was washed with dichloromethane. The filtrate was evaporated to afford the crude product which was purified firstly by MDAP followed by a further chromatographic purification (12 S silica column, ethyl acetate/isohohexane) to give the title compound (18.7%) MH+=312; ¹HNMR (DMSO, 250 MHz): 2.44 (3H s), 2.56 (3H s), 7.52 (2H m), 7.75 (4H m), 8.3 (1H d), 10.84 (1H s).

### Compound 23: N-(5-chloro-3-pyridinyl)-1-(4-fluorophenyl)-5-methyl-1H-1,2,3-triazole-4-carboxamide

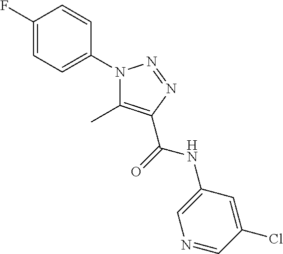

To a solution of 5-chloro-3-pyridinamine (25.8 mg, 0.201 mmol) in dry 1,4-dioxane (1 ml) was added dropwise trimethylaluminium (0.401 ml, 0.802 mmol) (2M solution in toluene). The solution was stirred at room temperature for 30 min. Then a solution of ethyl 1-(4-fluorophenyl)-5-methyl-1H-1,2,3-triazole-4-carboxylate (50 mg, 0.201 mmol) (Intermediate 2) in dry 1,4-dioxane (1 ml) was added at room temperature and the reaction was heated to 100° C. overnight. The reaction was cooled to room temperature, quenched cautiously with water (0.06 ml), and stirred at room temperature for 10 min. Then, sodium sulphate (0.25 g) was added to bind the water and the mixture was stirred vigorously for 5 min. Dichloromethane was added and the mixture was stirred for an additional 10 min at room temperature. The solution was filtered and the solid was washed with dichloromethane. The filtrate was evaporated to afford a residue which was partially dissolved into methanol. The solution was filtered and the solid washed with diethyl ether. The product was purified twice by MDAP to give the title compound; MH+=332; ¹HNMR (CDCl₃, 250 MHz): 2.67 (3H s), 7.31 (2H m), 7.49 (2H m), 8.37 (1H d), 8.47 (1H t), 8.63 (1H d), 9.15 (1H s).

### Compound 24: 1-(3,5-Difluorophenyl)-5-methyl-N-(2-methyl-4-pyrimidinyl)-1H-1,2,3-triazole-4-carboxamide

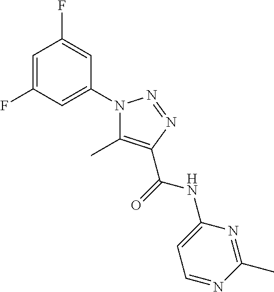

To a solution of 2-methyl-4-pyrimidinamine (0.059 g, 0.537 mmol) in dry 1,4-dioxane (1 ml) was added dropwise trimethylaluminium (0.262 ml, 0.524 mmol) (2M solution in toluene). The solution was stirred at room temperature for 30 min then a solution of ethyl 1-(3,5-difluorophenyl)-5-methyl-1H-1,2,3-triazole-4-carboxylate (0.035 g, 0.131 mmol) (Intermediate 4) in dry dioxane (1 ml) was added at room temperature and the reaction was heated to 100° C. for 1.5 hours. The reaction was cooled to room temperature quenched cautiously with water (0.2 ml) and stirred at room temperature for 10 min. The solvent was evaporated and the mixture was dissolved in dichloromethane and filtered. Evaporation of the filtrate gave the crude product that was purified by silica chromatography (12S column, EtOAc-isohex) to afford the title compound (20.1%); MH+=331; ¹HNMR (CDCl₃, 400 MHz): 2.67 (3H s), 2.75 (3H, s), 7.09 (3H m), 8.09 (1H d), 8.59 (1H d), 9.62 (1H s).

### Compound 25: 1-(3-Chloro-4-fluorophenyl)-5-methyl-N-(2-methyl-4-pyrimidinyl)-1H-1,2,3-triazole-4-carboxamide

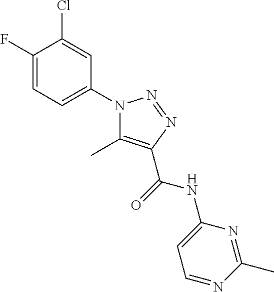

To a solution of 2-methyl-4-pyrimidinamine (0.060 g, 0.549 mmol) in dry 1,4-dioxane (1 ml) was added dropwise trimethylaluminium (0.268 ml, 0.536 mmol) (2M solution in toluene). The solution was stirred at room temperature for 30 min. Then a solution of ethyl 1-(3-chloro-4-fluorophenyl)-5-methyl-1H-1,2,3-triazole-4-carboxylate (0.038 g, 0.134 mmol) (Intermediate 5) in dry 1,4-dioxane (1 ml) was added at room temperature and the reaction was heated to 100° C. for 1.5 hours. The reaction was cooled to room temperature, quenched cautiously with water (0.06 ml), and stirred at room temperature for 10 min. Then, sodium sulphate (0.25 g) was added to bind the water and the mixture was stirred vigorously for 5 min. Dichloromethane was added and the mixture was stirred for additional 10 min at room temperature. The solution was filtered and the solid was washed with dichloromethane. The combined filtrate and washings were evaporated to a residue which was purified by MDAP to afford the title compound (16.8%); MH+=347; ¹HNMR (DMSO, 250 MHz): 2.57 (3H s), 2.59 (3H, s), 7.76 (2H m), 8.0 (1H d), 8.09 (1H m), 8.65 (1H d), 10.21 (1H s).

### Compound 26: N-(3-cyanophenyl)-1-(4-fluorophenyl)-5-methyl-1H-1,2,3-triazole-4-carboxamide

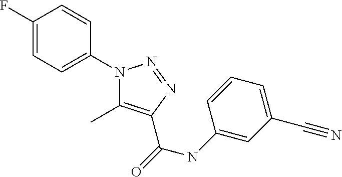

A solution of 1-(4-fluorophenyl)-5-methyl-1H-1,2,3-triazole-4-carboxylic acid [commercially available from ENAMINE Ltd. (23 Alexandra Matrosova Street, 01103 KIEV, Ukraine] (50 mg, 0.226 mmol) in thionyl chloride (10 ml) was heated under reflux for 2 hours. The mixture was evaporated in vacuo to give a residue and chloroform (5 ml) was added. A solution of 3-aminobenzonitrile (32 mg, 0.271 mmol) and TEA (46 mg, 0.451 mmol) in chloroform (10 ml) was added dropwise and the reaction mixture was stirred at room temperature for 3 hours. The mixture was evaporated in vacuo to give a residue which was purified by column chromatography on silica gel (ethyl acetate/petroleum ether=1/5 to 1/1) to give the title compound (30 mg, 41.4%); MH+=322.0; ¹HNMR (300 MHz, CDCl₃): δ9.164 (1H, s), 8.240 (1H, s), 7.81 (1H, d), 7.43-7.50 (4H, m), 7.31 (2H, dd), 2.674 (3H, s).

### Compound 27: 1-(3,4-Dimethylphenyl)-5-methyl-N-(2-methyl-4-pyrimidinyl)-1H-1,2,3-triazole-4-carboxamide

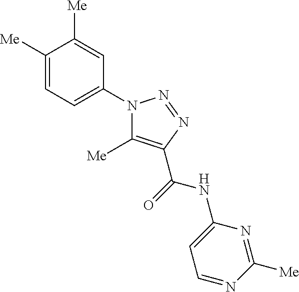

To a solution of 2-methyl-4-pyrimidinamine (0.047 g, 0.427 mmol) in dry 1,4-dioxane (1 ml) was added dropwise trimethylaluminium (0.208 ml, 0.417 mmol) (2M solution in toluene). The solution was stirred at room temperature for 30 min. Batches A and B of impure ethyl 1-(3,4-dimethylphenyl)-5-methyl-1H-1,2,3-triazole-4-carboxylate (0.013 g) (Intermediate 6) were dissolved in dry 1,4-dioxane (1 ml). This solution was added at room temperature to the reaction mixture and the mixture was heated to 100° C. overnight. The reaction was cooled to room temperature, quenched cautiously with water (0.06 ml), and stirred at room temperature for 10 min. Then, sodium sulphate (0.25 g) was added to bind the water and the mixture was stirred vigorously for 5 min. Dichloromethane was added and the mixture was stirred for an additional 10 min at room temperature. The solution was filtered and the solid was washed with dichloromethane. Evaporation of the solvent gave a crude residue which was purified by MDAP to afford the title compound (3.6 mg); MH+=323; ¹HNMR (CDCl₃, 250 MHz): 2.37 (6H s), 2.65 (3H, s), 2.67 (3H s), 7.22 (2H m), 7.33 (1H d), 8.09 (1H d), 8.57 (1H d), 9.67 (1H s).

### Compound 28: 1-(3-Chloro-4-fluorophenyl)-5-methyl-N-(2-methyl-4-pyridinyl)-1H-1,2,3-triazole-4-carboxamide

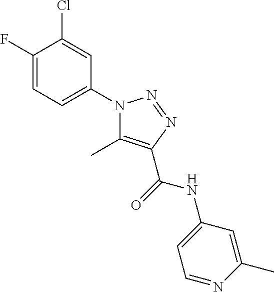

To a solution of 2-methyl-4-pyridinamine (64 mg, 0.226 mmol) in dry 1,4-dioxane (2 ml) was added dropwise trimethylaluminium (0.451 ml, 0.902 mmol) (2M solution in toluene). The solution was stirred at room temperature for 30 min. Then a solution of ethyl 1-(3-chloro-4-fluorophenyl)-5-methyl-1H-1,2,3-triazole-4-carboxylate (64 mg, 0.226 mmol) (Intermediate 5) in dry 1,4-dioxane (2 ml) was added at room temperature and the reaction was heated to 100° C. for 24 hours. The reaction was cooled to room temperature, quenched cautiously with water (0.12 ml), and stirred at room temperature for 10 min. Then, sodium sulphate (0.5 g) was added to bind the water and the mixture was stirred vigorously for 5 min. Dichloromethane was added and the mixture was stirred for an additional 10 min at room temperature. The solution was filtered and the solid was washed with dichloromethane. Evaporation of the solvent gave a crude residue which was purified by chromatography (12M silica column, ethylacetate-isohexane, 65%-35%) followed by MDAP to give the title compound (2.4%); MH+=346; ¹HNMR (CDCl₃, 400 MHz): 2.58 (3H s), 2.70 (3H, s), 7.40 (2H m), 7.44 (1H dd), 7.58-7.63 (2H m), 8.46 (1H d), 9.10 (1H s).

### Compound 29: 1-(3,4-Difluorophenyl)-5-methyl-N-(2-methyl-4-pyridinyl)-1H-1,2,3-triazole-4-carboxamide

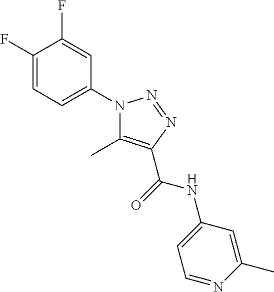

To a solution of 2-methyl-4-pyridinamine (166 mg, 1.534 mmol) in dry 1,4-dioxane (2 ml) was added dropwise trimethylaluminium (0.748 mL, 1.497 mmol) (2M solution in toluene). The solution was stirred at room temperature for 30 min. Then a solution of ethyl 1-(3,4-difluorophenyl)-5-methyl-1H-1,2,3-triazole-4-carboxylate (100 mg, 0.374 mmol) (Intermediate 7) in dry 1,4-dioxane (2.0 ml) was added at room temperature and the reaction was heated to 100° C. for 3 hours. The reaction was cooled to room temperature, quenched cautiously with water (0.125 ml), and stirred at room temperature for 10 min. Then, sodium sulphate (0.5 g) was added to bind the water and the mixture was stirred vigorously for 5 min. Dichloromethane was added and the mixture was stirred for an additional 10 min at room temperature. The solution was filtered and the solid was washed with dichloromethane. Evaporation of the solvent gave a crude product which was purified by chromatography (12M silica column, ethyl acetate-isohexane 50/50) to give the title compound (35.7%); MH+=330; ¹HNMR (DMSO, 400 MHz): 2.44 (3H s), 2.59 (3H, s), 7.61 (1H m), 7.68 (1H dd), 7.78 (2H m), 7.98 (1H m), 8.34 (1H d), 10.86 (1H s).

### Compound 30: N-(3-chlorophenyl)-1-(6-fluoro-3-pyridinyl)-5-methyl-1H-1,2,3-triazole-4-carboxamide

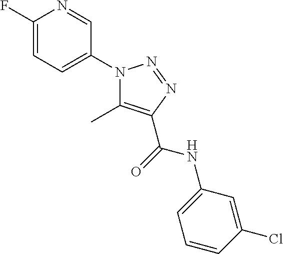

A mixture of 1-(6-fluoro-3-pyridinyl)-5-methyl-1H-1,2,3-triazole-4-carboxylic acid (0.05 g, 0.225 mmol) (Intermediate 9) and 3-chloroaniline (0.024 ml, 0.225 mmol) in N-methyl-2-pyrrolidone (NMP) (2 ml) and DIPEA (0.197 ml, 1.125 mmol) was stirred at room temperature. HOAt (0.061 g, 0.450 mmol) and HATU (0.171 g, 0.450 mmol) were then added. The mixture was stirred at room temperature for three days. The mixture was partitioned between ethyl acetate (20 ml) and saturated sodium bicarbonate solution (20 ml). The organic layer was separated, washed with water (20 ml) and brine (3×20 ml), dried (MgSO₄), filtered and concentrated under reduced pressure to give a crude residue. This residue was purified by MDAP to afford the title compound (54.7%); MH+=332; ¹HNMR (DMSO, 400 MHz): 2.60 (3H s), 7.17 (1H m), 7.38 (1H t), 7.56 (1H dd), 7.83 (1H m), 8.08 (1H t), 8.41 (1H m), 8.65 (1H d), 10.81 (1H s).

### Compound 31: N-(3-chlorophenyl)-1-(5-fluoro-2-pyridinyl)-5-methyl-1H-1,2,3-triazole-4-carboxamide

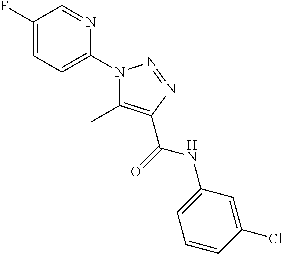

To a solution of 3-chloroaniline (0.017 ml, 0.164 mmol) in dry 1,4-dioxane (1 ml) was added dropwise trimethylaluminium (0.080 ml, 0.160 mmol) (2M solution in toluene). The solution was stirred at room temperature for 30 min, and then a solution of ethyl 1-(5-fluoro-2-pyridinyl)-5-methyl-1H-1,2,3-triazole-4-carboxylate (10 mg, 0.040 mmol) (Intermediate 10) in dry 1,4-dioxane (1 ml) was added at room temperature and the reaction was heated to 100° C. for 1.5 hours. The reaction was cooled to room temperature quenched cautiously with water (0.2 ml) and stirred at room temperature for 10 min. The solvent was evaporated and the residue was partially dissolved in methanol. Filtration gave the title compound as a solid (71.7%); MH+=332; ¹HNMR (DMSO, 400 MHz): 2.77 (3H s), 7.18 (1H m), 7.39 (1H t), 7.83 (1H m), 8.07 (2H m), 8.17 (1H m), 8.75 (1H d), 10.8 (1H s).

### Compound 32: N-(3-chlorophenyl)-5-methyl-1(6-methyl-3-pyridinyl)-1H-1,2,3-triazole-4-carboxamide

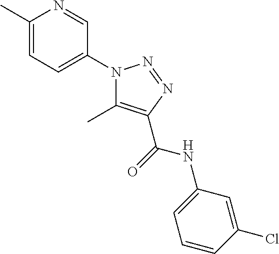

To a solution of 3-chloroaniline (0.021 ml, 0.200 mmol) in dry 1,4-dioxane (1 ml) was added dropwise trimethylaluminium (0.097 ml, 0.195 mmol) (2M solution in toluene). The solution was stirred at room temperature for 30 min, and then a solution of ethyl 5-methyl-1-(6-methyl-3-pyridinyl)-1H-1,2,3-triazole-4-carboxylate (0.012 g, 0.049 mmol) (Intermediate 11) in dry 1,4-dioxane (1 ml) was added at room temperature and the reaction was heated to 100° C. for 1.5 hours. The reaction was cooled to room temperature, quenched cautiously with water (0.2 ml), and stirred at room temperature for 10 min. Evaporation of the solvent gave a residue that was dissolved in dichloromethane and filtered. The filtrate was evaporated to give a crude product that was purified by silica chromatography (12S column, EtOAc-isohex) to afford the title compound (11.9%); MH+=328; ¹HNMR (CDCl₃, 400 MHz): 2.70 (3H s), 2.71 (3H, s), 7.14 (1H m), 7.31 (1H t), 7.42 (1H d), 7.50 (1H m), 7.74 (1H dd), 7.91 (1H t), 8.66 (1H d), 9.06 (1H s).

### Compound 33: 1(4-Fluorophenyl)-5-methyl-N-(4-methyl-2-pyrimidinyl)-1H-1,2,3-triazole-4-carboxamide

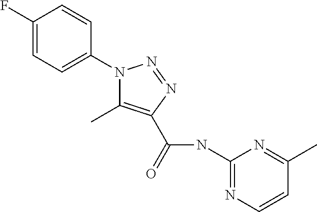

To a solution of 1-(4-fluorophenyl)-5-methyl-1H-1,2,3-triazole-4-carboxylic acid [commercially available from ENAMINE Ltd. (23 Alexandra Matrosova Street, 01103 KIEV, Ukraine] (50 mg, 0.23 mmol) in dichloromethane (10 ml) was added oxalyl chloride (58 mg, 0.46 mmol) slowly with ice-cooling. The mixture was allowed to warm to room temperature and stirred for 3 hrs. The solvent was evaporated and the resulting residue was dissolved in dichloromethane (5 ml). DIPEA (59 mg, 0.46 mmol) and 4-methyl-2-pyrimidinamine (38 mg, 0.35 mmol) were added and the mixture was stirred at room temperature for another 16 hrs. The mixture was then poured into water (50 ml). The mixture was extracted with EtOAc (2×50 ml) and the combined organic phases was concentrated under reduced pressure to give a residue. This residue was purified by preparative HPLC to give the title compound as a yellow solid (21 mg, 25%); MH+=313.1; ¹HNMR (300 MHz CDCl₃): δ 9.681 (s, 1H), 8.488-8.476 (d, 1H), 7.401-7.434 (m, 2H), 7.200-7.244 (m, 2H), 6.864-6.877 (t, 1H), 2.609 (s, 1H), 2.470 (s, 1H).

### Compound 34: N-(4-chloro-2-pyrimidinyl)-1-(4-fluorophenyl)-5-methyl-1H-1,2,3-triazole-4-carboxamide

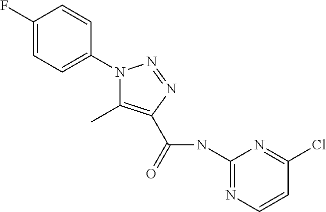

To a solution of 4-chloro-2-pyrimidinamine (88 mg, 0.68 mmol) in dry 1,4-dioxane (5 ml) was added dropwise trimethylaluminum (49 mg, 0.68 mmol) under an argon atmosphere. The solution was stirred at room temperature for 30 min. A solution of methyl 1-(4-fluorophenyl)-5-methyl-1H-1,2,3-triazole-4-carboxylate (Intermediate 8) (40 mg, 0.17 mmol) in 1,4-dioxane (3 ml) was then added. The reaction mixture was heated to 100° C. and stirred overnight. The solvent was evaporated in vacuo, the mixture poured into water (10 ml), and extracted with ethyl acetate (3×10 ml). The combined organic phases were dried over Na₂SO₄ and evaporated to afford a yellow solid. The solid was purified by preparative HPLC to obtain the title compound (15 mg, 21%); MH⁺=332.9; ¹HNMR (300 MHz CDCl₃): δ 9.844 (s, 1H), 8.579 (s, 1H), 7.461-7.484 (m, 2H), 7.260-7.312 (m, 2H), 7.109-7.112 (d, 1H), 2.668 (s, 1H).

### Compound 35: 1-(4-Fluorophenyl)-5-methyl-N-(6-methyl-4-pyrimidinyl)-1H-1,2,3-triazole-4-carboxamide

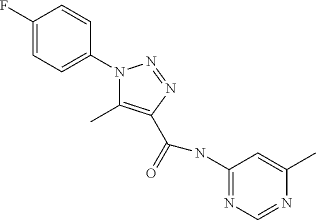

To a solution of 6-methyl-4-pyrimidinamine (93 mg, 0.85 mmol) in dry 1,4-dioxane (5 ml) was added dropwise trimethylaluminum (61 mg, 0.85 mmol) under an argon atmosphere and the solution was stirred at room temperature for 30 min. A solution of methyl 1-(4-fluorophenyl)-5-methyl-1H-1,2,3-triazole-4-carboxylate (Intermediate 8) (50 mg, 0.213 mmol) in 1,4-dioxane (3 ml) was then added and the reaction mixture was heated to 100° C. and stirred overnight. After cooling, the solvent was evaporated in vacuo and poured into water (10 ml). The aqueous mixture was then extracted with ethyl acetate (3×10 ml) and the combined organic phases were dried over Na₂SO₄. Evaporation gave a yellow solid, which was purified by preparative HPLC to give the title compound (40 mg, 61%); MH+=313.0; ¹HNMR (300 MHz CDCl₃): δ 9.660 (s, 1H), 8.826-8.829 (d, 1H), 8.177 (s, 1H), 7.466-7.500 (m, 2H), 7.263-7.327 (m, 2H), 2.673 (s, 3H), 2.566 (s, 3H).

### Compound 36: N-(6-chloro-4-pyrimidinyl)-1-(4-fluorophenyl)-5-methyl-1H-1,2,3-triazole-4-carboxamide

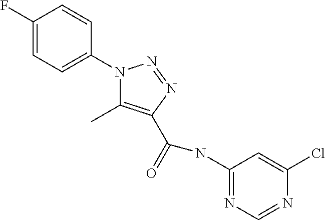

To a solution of 6-chloro-4-pyrimidinamine (110 mg, 0.85 mmol) in dry 1,4-dioxane (5 ml) was added dropwise trimethylaluminum (61 mg, 0.85 mmol) with Ar atmosphere. The mixture was stirred at r.t. for 30 min and then was added a solution of methyl 1-(4-fluorophenyl)-5-methyl-1H-1,2,3-triazole-4-carboxylate (Intermediate 8) (50 mg, 0.21 mmol) in 1,4-dioxane (3 ml). The reaction mixture was heated to 100° C. and stirred overnight. After cooling, the solvent was evaporated in vacuo and poured into water (10 ml), then extracted with ethyl acetate (3*10 ml). The combined organic phase was dried over Na₂SO₄ and evaporated to afford a yellow solid. The solid was purified by preparative HPLC to afford the title compound (35 mg, 49%), MH+=332.9; ¹HNMR (300 MHz CDCl₃): δ 9.781 (s, 1H), 8.727-8.7309 (d, 1H), 8.391-8.394 (d, 1H), 7.468-7.479 (m, 2H), 7.264-7.335 (m, 2H), 2.674 (s, 3H).

### Compound 37: 5-methyl-1-[3-(methyloxy)phenyl]-N-(6-methyl-2-pyridinyl)-1H-1,2,3-triazole-4-carboxamide

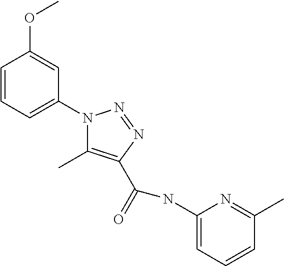

To a solution of 5-methyl-1-[3-(methyloxy)phenyl]-1H-1,2,3-triazole-4-carboxylic acid (100 mg, 0.43 mmol, commercially available from Akos) in DCM (5 ml) was added oxalyl chloride (109 mg, 0.86 mmol) slowly with ice-cooling. The mixture was allowed to warm up to room temperature and stirred for 2 hrs. The solvent was evaporated and the resulting residue was dissolved in dichloromethane (5 ml). DIPEA (111 mg, 0.86 mmol) and 6-methyl-2-pyridinamine (70 mg, 0.645 mmol) were added and the mixture was stirred at room temperature. for 3 hrs. The solvent was evaporated and the mixture was poured into water (50 ml). The aqueous mixture was extracted with EtOAc (2×50 ml) and the combined organic phases were dried and concentrated under reduced pressure to give a residue. This residue was purified by preparative HPLC to give the title compound as a white solid (55 mg, 25%); MH+=324.0; ¹HNMR (300 MHz CDCl₃): δ 9.622 (s, 1H), 8.137-8.158 (d, 1H), 7.623-7.660 (t, 1H), 7.461-7.502 (m, 1H), 7.106-7.113 (m, 1H), 7.021-7.093 (m, 2H), 6.932-7.089 (m, 1H), 3.884 (s, 1H), 2.691 (s, 1H), 2.515 (s, 1H).

### Compound 38: 5-methyl-1-[2-(methyloxy)phenyl]-N-(6-methyl-2-pyridinyl)-1H-1,2,3-triazole-4-carboxamide

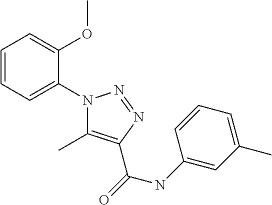

A mixture of 5-methyl-1-[2-(methyloxy)phenyl]-1H-1,2,3-triazole-4-carboxylic acid (100 mg, 0.43 mmol, commercially available from Akos) in thionyl chloride (10 ml) was heated under reflux for 2 h. The mixture was cooled to room temperature and the solvent was evaporated in vacuo. To the residue was added chloroform (5 ml). A solution of 6-methylpyridin-2-amine (51 mg, 0.473 mmol) and TEA (87 mg, 0.86 mmol) in chloroform (10 ml) was added dropwise and the reaction mixture was stirred at room temperature for 3 hours. The mixture was evaporated in vacuo to afford a yellow solid, which was purified by column chromatography on silica gel (ethyl acetate/petroleum ether=1/5 to 1/1) to afford the title compound (25 mg, 18%); MH+=324.0; ¹HNMR (300 MHz, CDCl₃): δ9.626 (1H, s), 8.142 (1H, d), 7.624 (1H, dd), 7.553 (1H, dd), 7.39 (1H, d), 7.1-7.16 (2H, m), 6.926 (1H, d), 3.824 (1H, s), 2.511 (1H, s), 2.501 (1H, s).

### Compound 39: N-(3-chlorophenyl)-5-[(methyloxy)methyl]-1-(4-methylphenyl)-1H-1,2,3-triazole-4-carboxamide

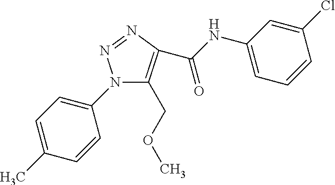

The title compound is commercially available from Ambinter SARL, 50 avenue de Versailles, 75016 Paris, France.

### Compound 40: 144-chlorophenyl)-N-(2,5-dichlorophenyl)-5-[(methyloxy)methyl]-1H-1,2,3-triazole-4-carboxamide

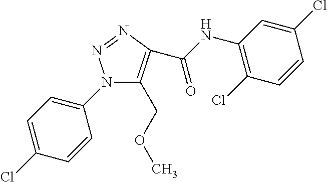

The title compound is commercially available from Ambinter SARL, 50 avenue de Versailles, 75016 Paris, France.

### Compound 41: 1-(3-chloro-4-fluorophenyl)-N-(3-chlorophenyl)-5-methyl-1H-1,2,3-triazole-4-carboxamide

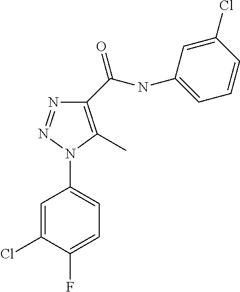

To a solution of 3-chloroaniline (0.119 ml, 1.128 mmol) in dry 1,4-dioxane (3 ml) was added dropwise trimethylaluminium (0.564 ml, 1.128 mmol) under an atmosphere of nitrogen. The solution was stirred at room temperature for 60 min and then a solution of ethyl 1-(3-chloro-4-fluorophenyl)-5-methyl-1H-1,2,3-triazole-4-carboxylate (Intermediate 5) (80 mg, 0.282 mmol) in 1,4-dioxane (3 ml) was added at room temperature. The reaction mixture was heated at 100° C. for 22 hrs. The reaction mixture was cooled to room temperature, quenched with water (1 ml) and stirred at room temperature for 10 min. The solvent was evaporated and the residue was dissolved in DCM (50 ml). The mixture was washed with water (2×10 ml), then brine (1×10 ml) and the organic phase filtered through a hydrophobic frit (Phase Separator cartridge). The filtered organic phase was evaporated under vacuum and the residue was purified via Biotage using a 25M column of silica, eluting with Cy:AcOEt (from 50 to 100%) to give the title compound (90 mg, 0.246 mmol, 87% yield); ¹H NMR (400 MHz, CHLOROFORM-d) δ : ppm 2.67-2.72 (m, 3H) 7.11-7.17 (m, 1H) 7.27-7.34 (m, 1H) 7.37-7.42 (m, 2H) 7.48-7.53 (m, 1H) 7.60-7.64 (m, 1H) 7.90 (t, J=2.05 Hz, 1H) 9.07 (s, 1H); UPLC-MS: 0.86 min, 365[M+H]+.

### EXAMPLE 42

### 1-(3-chloro-4-fluorophenyl)-N-(3-chlorophenyl)-5-methyl-1H-1,2,3-triazole-4-carbothioamide

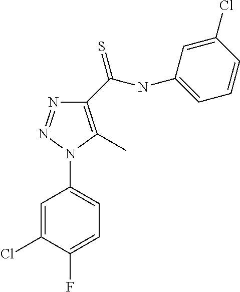

1-(3-Chloro-4-fluorophenyl)-N-(3-chlorophenyl)-5-methyl-1H-1,2,3-triazole-4-carboxamide (Compound 41) (70 mg, 0.192 mmol) was dissolved in toluene (3 ml) in a microwave reactor vial and Lawesson's reagent (78 mg, 0.192 mmol) was added. The reaction mixture was stirred and heated at 120° C. After 24 h further Lawesson's reagent was added (22 mg, 0.05 mmol) and the mixture was reheated at 120° C. overnight. The reaction mixture was quenched with water (10 ml) and the organic phase was extracted with DCM (2×50 ml). The organic layers were combined, filtered through a hydrophobic frit (Phase Separator cartridge), and evaporated under vacuum. The residue was purified via Biotage using a 25M column of silica eluted with Cy:AcOEt (from 0 to 100%) to give the title compound (48.7 mg, 0.128 mmol, 66.6% yield); ¹H NMR (400 MHz, CHLOROFORM-d) δ ppm 2.83 (s, 3H) 7.26-7.46 (m, 4H) 7.60-7.65 (m, 1H) 7.75-7.80 (m, 1H) 8.10 (t, 1H) 10.79 (br. s., 1H); UPLC-MS: 0.94 min, [MH+/−]=381.

### EXAMPLE 43

### 1-(3-chloro-4-fluorophenyl)-N-(2-chloro-4-pyridinyl)-5-methyl-1H-1,2,3-triazole-4-carboxamide

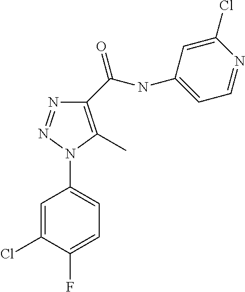

To a solution of 2-chloro-4-pyridinamine (22.66 mg, 0.176 mmol) in dry 1,4-dioxane (2 ml) was added dropwise trimethylaluminium (0.353 ml, 0.705 mmol) under an atmosphere of nitrogen. The solution was stirred at room temperature for 60 min and then a solution of ethyl 1-(3-chloro-4-fluorophenyl)-5-methyl-1H-1,2,3-triazole-4-carboxylate (Intermediate 5) (50 mg, 0.176 mmol) in 1,4-dioxane (2.000 ml) was added at room temperature. The reaction was heated at 100° C. for 22 hrs. The reaction was cooled to room temperature, quenched with water (1 ml) and stirred at room temperature for 10 min. The solvent was evaporated and the residue was dissolved in DCM (50 ml). The mixture was washed with water (2×10 ml), then brine (1×10 ml) and the organic phase filtered through a hydrophobic frit (Phase Separator cartridge) and the solvent evaporated under vacuum. The residue was purified via Biotage using a 25M column of silica eluting with Cy:AcOEt (from 50 to 100%) to give the title compound (44 mg, 0.120 mmol, 68.2% yield); ¹H NMR (400 MHz, CHLOROFORM-d) δ ppm 2.70 (s, 3H) 7.40-7.43 (m, 2H) 7.51 (dd, J=5.62, 1.96 Hz, 1H) 7.61-7.64 (m, 1H) 7.87-7.89 (m, 1H) 8.32-8.35 (m, 1H) 9.24 (s, 1H); UPLC-MS: 0.92 min; [MH+/−]=366.

The supporting compounds were tested in one of the following calcium mobilisation assays.

### a) FLIPR Assay

### Cell Preparation

24 hrs prior to running the assay, Chinese hamster ovary (CHO) cells containing human mGluR5 receptors engineered with geneswitch expression control technology (supplied by Invitrogen) were prepared. These cells were induced with 0.1 nM mifepristone to enable expression and growth media was added. The growth media comprised DMEM (Dulbeccos Modifies Eagle medium) 500 mls (supplied by Gibco—catalogue number 31166), 50 ml dialysed FCS (Foetal Calf Serum) (supplied by Gibco—catalogue number 26400-044), 0.02 mg/ml proline, 0.25 mg/ml hygromycin and 0.5 mg/ml zeocin. The cells in the growth media were then dispensed using a Multidrop (supplied by ThermoFisher) into 384-well black clear-bottom plates at a confluency of 10K cells/well in a volume of 50 μl. The plates were incubated at 37° C. to give a monolayer with a confluency of 80%.

On the day of the assay, the growth medium was aspirated using a power washer (supplied by Tecan) and 30 μl of loading buffer was added to the cells using a Multidrop. The loading buffer comprised Hanks Balanced Salt Solution (HBSS)+2.5M probenicid+2 μM Fluo-4 (MDC) and 250 μM Brilliant Black (MDC). The cells were incubated at 37° C. for 2 hours (This allows uptake of the dye Fluo-4 AM into the cell cytosol where it becomes trapped when natural esterases cleave the AM region of the Fluo-4. In the event of a response to ligand at the mGluR5 receptors, calcium will move from the internal stores in the endoplasmic reticulum into the cytosol where it will bind to the Fluo-4 dye and fluoresce when excited by laser light).

### Compound Preparation

The test compounds, in DMSO at a concentration of 10 mM, were further diluted with DMSO using a Biomek FX (supplied by Beckman Coulter) into a 384-well compound plate (supplied by Greiner). Each dilution was then transferred in 1 ul aliquots to a further compound plate and assay buffer added to give a final volume of 50 μl, making a final assay concentration of 11.8 μM. The assay buffer consisted of HBSS and 2.5M probenicid.

### Glutamate Dose Response Curve Preparation

A 0.38M solution of glutamic acid in water, was further diluted in DMSO to a concentration of 204 mM. 16×11 point concentration response curves (CRCs) were prepared in DMSO with a top concentration of 204 mM, making the final assay concentration 1 mM, diluted serially to 1 in 4. This was performed using the Biomek FX liquid handling device. 1 μl stampouts of the CRCs were prepared. 50 μl of compound buffer was added prior to use.

### Estimation of EC80 Concentration of Glutamate (Endogenous Ligand)

50 μl of compound buffer was added to the glutamate CRC plate. This plate was added using the FLIPR to a plate of cells that had been loaded for 2 hrs. A fluorescent read was made. An EC80 concentration of glutamate was calculated by using 4×EC50 concentration generated. An EC80 concentration solution of glutamate was prepared in compound buffer and dispensed into 384-well plates, excluding column 18 where compound buffer alone was added.

### Running the Assay

10 μl of test compounds were added to the cell plates using a Cybiwell liquid handling device (supplied by Cybio). The cell plates were incubated at 37° C. for 15 min, a FLI PR addition of the EC80 was made and a fluorescent read generated. Blocking of the receptor by test compound, in a dose dependent manner is evident from the calcium vs time profiles generated for each well. The data is analysed using XC50 software to produce CRCs, from which the potency and pIC50 can be determined.

b) mGluR5 Aequorin Assay

### Cell Preparation

CHO cells containing human mGluR5 receptors with Tet On expression control technology (supplied by Clontech) were prepared. These cells were grown in cell factories, induced with 10 ng/ml doxycycline to enable expression, harvested and then cryo-preserved at −140° C. in 1 ml aliquots for future use.

On the afternoon prior to the assay, the cells were thawed, suspended in growth media and centrifuged at 1000 rpm for 5 min. The growth media consisted of F12 Hams Nutrient mix (supplied by Gibco—catalogue number 21765) and 10% Tet approved FBS (supplied by Clontech—catalogue number 631106). The cells were then re-suspended in growth media and incubated at 37° C. for 1 hour in a spinner flask. After this post thaw recovery period, the cell suspension was centrifuged once more and resuspended at 2.5×10⁶ cells/ml in loading buffer consisting of HBSS, 0.1% BSA (supplied by CalBiochem—catalogue number 126609) and 0.1% Pluronic F68 (supplied by Gibco—catalogue number 24040-032). The cells were loaded with coelentrazine (supplied by Invitrogen C—catalogue number 6780) to a concentration of 5 μM, wrapped in foil and loaded overnight with mixing. Immediately prior to the assay, the cells were diluted to 15×10⁵ cells/ml in dilution buffer consisting of HBSS and 0.1% Pluronic F68.

Coelentrazine is the chromophore co-factor which activates the apo-protein, aequorin. The protein has three high affinity binding sites for calcium. Upon agonism of the mGluR5 receptor, binding of calcium to the aequorin protein induces a conformational change resulting in an oxidative decarboxylation reaction producing coelenteramide and a flash luminescence signal. This signal was measured using the Lumilux (supplied by Perkin Elmer).

### Compound Preparation

The test compounds were prepared in DMSO at a concentration of 3 mM. These solutions are serially diluted with DMSO to 1 in 4 using a Biomek FX liquid handling device (supplied by Beckman Coulter) in a 384-well compound plate (supplied by Greiner). Daughter plates of 0.5 μl/well were stamped-out from this master plate for use in the assay.

### Glutamate Dose Response Curve Preparation

A 100 mM solution of glutamic acid was prepared in water. This was further diluted with DMSO to a concentration of 10 mM. 16×11 point concentration response curves (CRC) were prepared in DMSO, making the final assay concentration 1.66×10⁻⁴M, with 1 in 3 serial dilutions using the Biomek FX. 0.5 μl stamp-outs of this plate were generated for use in the assay.

### Estimation of EC80 Concentration of Glutamate

The glutamate CRC plate was placed on the Lumilux where 20 μl/well of dilution buffer was added, followed by 10 μl/well of loaded cell suspension and a luminescence read was made. An EC80 concentration of glutamate was calculated by using 4×EC50 generated. The EC80 solution was prepared in dilution buffer and added to a reservoir within the Lumilux.

### Running the Assay

20 μl/well of buffer and 10 μl/well of cell suspension were added to the test compounds using the lumilux. These plates were incubated at room temperature for 15 mins, 10 μl/well of EC80 solution added and a luminescence read made. Blocking of the receptor by test compound in a dose dependent manner was evident from the luminescence vs time profiles generated for each well. The data was analysed using XC50 software to produce CRCs, from which the potency and pIC50 can be determined.

The Supporting Compounds have been tested in assay b). Using assay b), compounds 1 to 39 and 41 to 43 gave a pIC50 between 4.9 and 9.0.

1. A compound of formula (I) or a pharmaceutically acceptable salt thereof, for use in therapy, 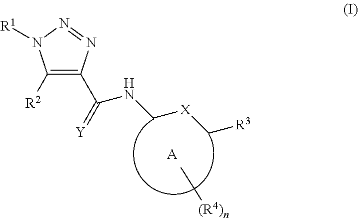
wherein
R¹ is phenyl or 6-membered monocyclic heteroaryl, either of which are optionally substituted by one, two or three groups independently selected from halo, C₁₋₆alkyl, C₁₋₆alkoxy, C₁₋₆haloalkyl, C₁₋₆haloalkoxy, cyano, C₁₋₆alkylthio, C₁₋₆haloalkylthio, C₁₋₆alkylsulfonyl, C₁₋₆haloalkylsulfonyl, C₁₋₆alkylcarbonyl and C₁₋₆haloalkylcarbonyl;
R² is halo, C₁₋₆alkyl, C₁₋₆alkoxy, C₁₋₆haloalkyl, C₁₋₆haloalkoxy or C₁₋₄alkoxyC₁₋₄alkyl;
Y is O or S;
A is either a 6-membered heteroaromatic ring or a benzene ring; wherein X is CH, N, S or O; and wherein when A is a 6-membered heteroaromatic ring, the ring may contain a total of 1 to 4 heteroatoms independently selected from N, O and S;
R³ is halo, C₁₋₆alkyl, C₁₋₆alkoxy, C₁₋₆haloalkyl, C₁₋₆haloalkoxy, C₁₋₄alkoxyC₁₋₄alkyl or cyano;
R⁴ is halo; and
n is 0 or 1.

2. A compound according to claim 1 or a pharmaceutically acceptable salt thereof wherein R¹ is substituted by one or two groups independently selected from halo, C₁₋₆alkyl, C₁₋₆alkoxy, C₁₋₆haloalkyl, C₁₋₆haloalkoxy, cyano, C₁₋₆alkylthio, C₁₋₆haloalkylthio, C₁₋₆alkylsulfonyl, C₁₋₆haloalkylsulfonyl, C₁₋₆alkylcarbonyl and C₁₋₆haloalkylcarbonyl.

3. A compound according to claim 1 or a pharmaceutically acceptable salt thereof wherein R¹ is phenyl or pyridyl either of which are optionally substituted by one or two groups independently selected from halo, C₁₋₆alkyl, C₁₋₆alkoxy, C₁₋₆haloalkyl, C₁₋₆haloalkoxy, C₁₋₆alkylthio and C₁₋₆haloalkylthio.

4. A compound according to claim 1 or a pharmaceutically acceptable salt thereof wherein R² is C₁₋₆alkyl or C₁₋₄alkoxyC₁₋₄alkyl.

5. A compound according to claim 1 or a pharmaceutically acceptable salt thereof wherein Y is O.

6. A compound according to claim 1 or a pharmaceutically acceptable salt thereof wherein A is 6-membered heteroaromatic ring; wherein X is CH or N; and wherein the ring may contain a total of 1 to 4 nitrogen atoms.

7. A compound according to claim 1 or a pharmaceutically acceptable salt thereof wherein A is 6-membered heteroaromatic ring; wherein X is CH or N; and wherein the ring contains a total of 1 or 2 nitrogen atoms.

8. A compound according to claim 1 or a pharmaceutically acceptable salt thereof wherein A is a pyridine ring.

9. A compound according to claim 1 or a pharmaceutically acceptable salt thereof wherein A is a pyridine ring attached to the amide nitrogen in formula (I) at the 4-position, i.e. having the following structure where the arrow indicates the point of attachment.

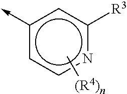

10. A compound according to claim 1 or a pharmaceutically acceptable salt thereof wherein R³ is halo, C₁₋₆alkyl, C₁₋₆haloalkyl or cyano.

11. A compound according to claim 1 or a pharmaceutically acceptable salt thereof wherein n is 0.

12. A compound according to claim 1 wherein the compound of formula (I) is selected from:
1-(4-fluorophenyl)-5-methyl-N-(6-methyl-2-pyridinyl)-1H-1,2,3-triazole-4-carboxamide (Compound 1);
1-(4-fluorophenyl)-5-methyl-N-(2-methyl-4-pyrimidinyl)-1H-1,2,3-triazole-4-carboxamide (Compound 2);
N-(3-chlorophenyl)-5-methyl-1-[3-(methylthio)phenyl]-1H-1,2,3-triazole-4-carboxamide (Compound 4);
N-(3-chlorophenyl)-1-(2,5-difluorophenyl)-5-methyl-1H-1,2,3-triazole-4-carboxamide (Compound 5):
N-(3-chlorophenyl)-5-methyl-1-phenyl-1H-1,2,3-triazole-4-carboxamide (Compound 6);
N-(3-chlorophenyl)-1-(4-fluorophenyl)-5-methyl-1H-1,2,3-triazole-4-carboxamide (Compound 7);
N-(3-fluorophenyl)-1-(4-fluorophenyl)-5-methyl-1H-1,2,3-triazole-4-carboxamide (Compound 8);
N-(3-chlorophenyl)-5-methyl-1-(2-methylphenyl)-1H-1,2,3-triazole-4-carboxamide (Compound 9);
N-(3-chlorophenyl)-1-(2-fluorophenyl)-5-methyl-1H-1,2,3-triazole-4-carboxamide (Compound 10);
1-(3-chloro-4-fluorophenyl)-N-(3-fluorophenyl)-5-methyl-1H-1,2,3-triazole-4-carboxamide (Compound 11);
N-(3-chlorophenyl)-1-(3-fluorophenyl)-5-methyl-1H-1,2,3-triazole-4-carboxamide (Compound 13);
N-(3-chlorophenyl)-1-(3,4-dimethylphenyl)-5-methyl-1H-1,2,3-triazole-4-carboxamide (Compound 14)
1-(4-fluorophenyl)-N-(6-fluoro-2-pyridinyl)-5-methyl-1H-1,2,3-triazole-4-carboxamide (Compound 20);
N-(6-chloro-2-pyridinyl)-1-(4-fluorophenyl)-5-methyl-1H-1,2,3-triazole-4-carboxamide (Compound 21);
1-(4-fluorophenyl)-5-methyl-N-(2-methyl-4-pyridinyl)-1H-1,2,3-triazole-4-carboxamide (Compound 22);
1-(3-chloro-4-fluorophenyl)-5-methyl-N-(2-methyl-4-pyrimidinyl)-1H-1,2,3-triazole-4-carboxamide (Compound 25);
N-(3-cyanophenyl)-1-(4-fluorophenyl)-5-methyl-1H-1,2,3-triazole-4-carboxamide (Compound 26);
1-(3,4-dimethylphenyl)-5-methyl-N-(2-methyl-4-pyrimidinyl)-1H-1,2,3-triazole-4-carboxamide (Compound 27);
1-(3-chloro-4-fluorophenyl)-5-methyl-N-(2-methyl-4-pyridinyl)-1H-1,2,3-triazole-4-carboxamide (Compound 28);
1-(3,4-difluorophenyl)-5-methyl-N-(2-methyl-4-pyridinyl)-1H-1,2,3-triazole-4-carboxamide (Compound 29); and
N-(3-chlorophenyl)-1-(5-fluoro-2-pyridinyl)-5-methyl-1H-1,2,3-triazole-4-carboxamide (Compound 31); or a pharmaceutically acceptable salt of any of the compounds.

13. A compound according to claim 1 wherein the compound of formula (I) is selected from:
1-(4-fluorophenyl)-5-methyl-N-(6-methyl-2-pyridinyl)-1H-1,2,3-triazole-4-carboxamide (Compound 1);
1-(4-fluorophenyl)-5-methyl-N-(2-methyl-4-pyrimidinyl)-1H-1,2,3-triazole-4-carboxamide (Compound 2);
1-(4-fluorophenyl)-N-(6-fluoro-2-pyridinyl)-5-methyl-1H-1,2,3-triazole-4-carboxamide (Compound 20);
N-(6-chloro-2-pyridinyl)-1-(4-fluorophenyl)-5-methyl-1H-1,2,3-triazole-4-carboxamide (Compound 21);
1-(4-fluorophenyl)-5-methyl-N-(2-methyl-4-pyridinyl)-1H-1,2,3-triazole-4-carboxamide (Compound 22);
1-(3-chloro-4-fluorophenyl)-5-methyl-N-(2-methyl-4-pyrimidinyl)-1H-1,2,3-triazole-4-carboxamide (Compound 25);
1-(3,4-dimethylphenyl)-5-methyl-N-(2-methyl-4-pyrimidinyl)-1H-1,2,3-triazole-4-carboxamide (Compound 27);
1-(3-chloro-4-fluorophenyl)-5-methyl-N-(2-methyl-4-pyridinyl)-1H-1,2,3-triazole-4-carboxamide (Compound 28); and
1-(3,4-difluorophenyl)-5-methyl-N-(2-methyl-4-pyridinyl)-1H-1,2,3-triazole-4-carboxamide (Compound 29); or a pharmaceutically acceptable salt of any of the compounds.

14. A compound of formula (I) or a pharmaceutically acceptable salt thereof 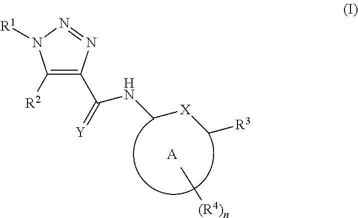
wherein
R¹ is phenyl or 6-membered monocyclic heteroaryl, either of which are optionally substituted by one or two groups independently selected from halo, C₁₋₆alkyl, C₁₋₆alkoxy, C₁₋₆haloalkyl, C₁₋₆haloalkoxy, cyano, C₁₋₆alkylthio, C₁₋₆haloalkylthio, C₁₋₆alkylsulfonyl, C₁₋₆haloalkylsulfonyl, C₁₋₆alkylcarbonyl and C₁₋₆haloalkylcarbonyl;
R² is halo, C₁₋₆alkyl, C₁₋₆alkoxy, C₁₋₆haloalkyl, C₁₋₆haloalkoxy or C₁₋₄alkoxyC₁₋₄alkyl;
Y is O;
A is a 6-membered heteroaromatic ring; wherein X is CH, N, S or O; and wherein when A is a 5 or 6-membered heteroaromatic ring, the ring may contain a total of 1 to 4 heteroatoms independently selected from N, O and S;
R³ is halo, C₁₋₆alkyl, C₁₋₆alkoxy, C₁₋₆haloalkyl, C₁₋₆haloalkoxy, or cyano;
R⁴ is halo; and
n is 0 or 1.

15. A compound according to claim 14 or a pharmaceutically acceptable salt thereof wherein R¹ is phenyl or pyridyl either of which are optionally substituted by one or two groups independently selected from halo, C₁₋₆alkyl, C₁₋₆alkoxy, C₁₋₆haloalkyl, C₁₋₆haloalkoxy, C₁₋₆alkylthio and C₁₋₆haloalkylthio.

16. A compound according to claim 14 or a pharmaceutically acceptable salt thereof wherein R² is C₁₋₆alkyl or C₁₋₄alkoxyC₁₋₄alkyl.

17. A compound according to claim 16 or a pharmaceutically acceptable salt thereof wherein R² is C₁₋₆alkyl.

18. A compound according to claim 14 or a pharmaceutically acceptable salt thereof wherein A is 6-membered heteroaromatic ring; wherein X is CH or N; and wherein the ring may contain a total of 1 to 4 nitrogen atoms.

19. A compound according to claim 14 or a pharmaceutically acceptable salt thereof wherein A is 6-membered heteroaromatic ring; wherein X is CH or N; and wherein the ring contains a total of 1 or 2 nitrogen atoms.

20. A compound according to claim 14 or a pharmaceutically acceptable salt thereof wherein A is a pyridine ring.

21. A compound according to claim 14 or a pharmaceutically acceptable salt thereof, wherein A is a pyridine ring attached to the amide nitrogen in formula (I) at the 4-position, i.e. having the following structure where the arrow indicates the point of attachment.

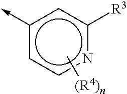

22. A compound according to claim 14 or a pharmaceutically acceptable salt thereof, wherein R³ is halo, C₁₋₆alkyl, C₁₋₆haloalkyl or cyano.

23. A compound according to claim 14 or a pharmaceutically acceptable salt thereof wherein n is 0.

24. A compound according to claim 14 selected from:
1-(4-fluorophenyl)-5-methyl-N-(6-methyl-2-pyridinyl)-1H-1,2,3-triazole-4-carboxamide (Compound 1);
1-(4-fluorophenyl)-5-methyl-N-(2-methyl-4-pyrimidinyl)-1H-1,2,3-triazole-4-carboxamide (Compound 2);
1-(4-fluorophenyl)-N-(6-fluoro-2-pyridinyl)-5-methyl-1H-1,2,3-triazole-4-carboxamide (Compound 20);
N-(6-chloro-2-pyridinyl)-1-(4-fluorophenyl)-5-methyl-1H-1,2,3-triazole-4-carboxamide (Compound 21);
1-(4-fluorophenyl)-5-methyl-N-(2-methyl-4-pyridinyl)-1H-1,2,3-triazole-4-carboxamide (Compound 22);
1-(3-chloro-4-fluorophenyl)-5-methyl-N-(2-methyl-4-pyrimidinyl)-1H-1,2,3-triazole-4-carboxamide (Compound 25);
1-(3,4-dimethylphenyl)-5-methyl-N-(2-methyl-4-pyrimidinyl)-1H-1,2,3-triazole-4-carboxamide (Compound 27);
1-(3-chloro-4-fluorophenyl)-5-methyl-N-(2-methyl-4-pyridinyl)-1H-1,2,3-triazole-4-carboxamide (Compound 28); and
1-(3,4-difluorophenyl)-5-methyl-N-(2-methyl-4-pyridinyl)-1H-1,2,3-triazole-4-carboxamide (Compound 29); or a pharmaceutically acceptable salt of any of the compounds.

25. A compound of formula (I) or a pharmaceutically acceptable salt thereof, 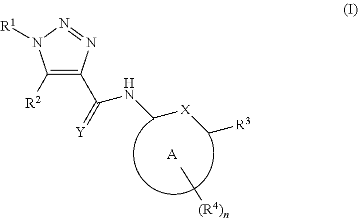
wherein
R¹ is a 6-membered monocyclic heteroaryl optionally substituted by one or two groups independently selected from halo, C₁₋₆alkyl, C₁₋₆alkoxy, C₁₋₆haloalkyl, C₁₋₆haloalkoxy, cyano, C₁₋₆alkylthio, C₁₋₆haloalkylthio, C₁₋₆alkylsulfonyl, C₁₋₆haloalkylsulfonyl, C₁₋₆alkylcarbonyl and C₁₋₆haloalkylcarbonyl;
R² is halo, C₁₋₆alkyl, C₁₋₆alkoxy, C₁₋₆haloalkyl, C₁₋₆haloalkoxy or C₁₋₄alkoxyC₁₋₄alkyl;
Y is O;
A is either a 6-membered heteroaromatic ring or a benzene ring; wherein X is CH, N, S or O; and wherein when A is a 6-membered heteroaromatic ring, the ring may contain a total of 1 to 4 heteroatoms independently selected from N, O and S;
R³ is halo, C₁₋₆alkyl, C₁₋₆alkoxy, C₁₋₆haloalkyl, C₁₋₆haloalkoxy, C₁₋₄alkoxyC₁₋₄alkyl or cyano;
R⁴ is halo; and
n is 0 or 1.

26. A compound according to claim 25 or a pharmaceutically acceptable salt thereof wherein R¹ is pyridyl optionally substituted by one or two groups independently selected from halo, C₁₋₆alkyl, C₁₋₆alkoxy, C₁₋₆haloalkyl, C₁₋₆haloalkoxy, C₁₋₆alkylthio and C₁₋₆haloalkylthio.

27. A compound according to claim 25 or a pharmaceutically acceptable salt thereof wherein R² is C₁₋₆alkyl or C₁₋₄alkoxyC₁₋₄alkyl.

28. A compound according to claim 27 or a pharmaceutically acceptable salt thereof wherein R² is C₁₋₆alkyl.

29. A compound according to claim 25 or a pharmaceutically acceptable salt thereof wherein A is 6-membered heteroaromatic ring; wherein X is CH or N; and wherein the ring may contain a total of 1 to 4 nitrogen atoms.

30. A compound according to claim 29 or a pharmaceutically acceptable salt thereof wherein A is 6-membered heteroaromatic ring; wherein X is CH or N; and wherein the ring contains a total of 1 or 2 nitrogen atoms.

31. A compound according to claim 30 or a pharmaceutically acceptable salt thereof wherein A is a pyridine ring.

32. A compound according to claim 31 or a pharmaceutically acceptable salt thereof wherein A is a pyridine ring attached to the amide nitrogen in formula (I) at the 4-position, i.e. having the following structure where the arrow indicates the point of attachment.

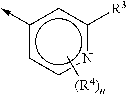

33. A compound according to claim 25 or a pharmaceutically acceptable salt thereof wherein R³ is halo, C₁₋₆alkyl, C₁₋₆haloalkyl or cyano.

34. A compound according to claim 25 or a pharmaceutically acceptable salt thereof wherein n is 0.

35. A compound according to claim 25 which is N-(3-chlorophenyl)-1-(5-fluoro-2-pyridinyl)-5-methyl-1H-1,2,3-triazole-4-carboxamide or a pharmaceutically acceptable salt thereof.

36-38. (canceled)

38. A method of treating a substance related disorder in a human which comprises administering an effective amount of a compound defined in claim 1 or a pharmaceutically acceptable salt thereof.

39. A pharmaceutical composition comprising a) a compound defined in claim 1 or a pharmaceutically acceptable salt thereof of the invention and b) one or more pharmaceutically-acceptable excipients.
